SEQUENCE LISTING (134 sequences)

SEQ ID NO: 1 - protein, 114 aa, synthetic construct
  REGION 1..114: note = Synthetic: SD-233883_VH
EVQLQQFGAELVKPGASVKLSCKASGYTFTSYDINWVRQRPEQGLEWIGWIFPGDGTSKYNEKFKGKATLSTDKSSSTAYMQLSRLTIEDSAVYFCARRLGSHWGQGSTLTVSS

SEQ ID NO: 2 - protein, 111 aa, synthetic construct
  REGION 1..111: note = Synthetic: SD-233883_VL
DIVLTQSPASLAVSLGQRATISCKASQSVDSDGDSYMNWYQQKPGQPPKLLIYGASNLESGIPARFSGSGSGTDFSLNIHPVEEEDAATYYCQQSHEYPFTFGGGTKLEIK

SEQ ID NO: 3 - protein, 8 aa, synthetic construct
  REGION 1..8: note = Synthetic: SD-233883_hCDR1
GYTFTSYD

SEQ ID NO: 4 - protein, 8 aa, synthetic construct
  REGION 1..8: note = Synthetic: SD-233883_hCDR2
IFPGDGTS

SEQ ID NO: 5 - protein, 7 aa, synthetic construct
  REGION 1..7: note = Synthetic: SD-233883_hCDR3
ARRLGSH

SEQ ID NO: 6 - protein, 10 aa, synthetic construct
  REGION 1..10: note = Synthetic: SD-233883_lCDR1
QSVDSDGDSY

SEQ ID NO: 7 - skipped

SEQ ID NO: 8 - protein, 9 aa, synthetic construct
  REGION 1..9: note = Synthetic: SD-233883_lCDR3
QQSHEYPFT

SEQ ID NO: 9 - DNA, 342 bp, synthetic construct
  misc_feature 1..342: note = Synthetic: SD-233883_VH_DNA
  source 1..342: mol_type = other DNA
gaagtgcaactccagcaattcggtgccgagcttgtgaagcctggggcctcagtgaagctgagctgcaaggcctccggatataccttcacctcctacgatatcaactgggtccggcagaggccggaacagggcctggagtggatcggttggatcttccccggcgacgggacctcgaagtacaacgaaaagttcaagggaaaagcaacgctgtccaccgacaagtcctcatccactgcgtacatgcagctgtcccgcctgactattgaggactccgctgtgtacttttgtgcccggagactcggaagccactggggacagggcagcaccttgactgtgtcgtcg

SEQ ID NO: 10 - DNA, 333 bp, synthetic construct
  misc_feature 1..333: note = Synthetic: SD-233883_VL_DNA
  source 1..333: mol_type = other DNA
gatattgtgctgactcaaagccctgcgtccctggctgtctccctgggacagcgcgccaccatttcatgcaaagcctcccagtccgtggacagcgacggggacagctatatgaactggtaccagcagaagcccgggcagcctccgaagctgcttatctacggtgcctccaacttggagtcgggaatccccgcacggttctccggatcgggctccggaactgacttctcgctcaacatccacccagtggaagaagaagatgccgccacctactactgtcagcaatcacatgagtacccgtttaccttcggtggcggcaccaagctcgagatcaag

SEQ ID NO: 11 - protein, 114 aa, synthetic construct
  REGION 1..114: note = Synthetic: SD-633416_VH
EVQLVQSGAEVKKPGASVKVSCKASGYTFTSYDINWVRQAPGQGLEWMGWIFPGDGTSKYAQKFQGRVTMTRDTSTSTVYMELSSLRSEDTAVYYCARRLGSHWGQGTTVTVSS

SEQ ID NO: 12 - protein, 111 aa, synthetic construct
  REGION 1..111: note = Synthetic: SD-633416_VL
DIQMTQSPSSLSASVGDRVTITCKASQSVDSDGDSYMNWYQQKPGKAPKLLIYGASNLESGVPSRFSGSGSGTDFTLTISSLQPEDFATYYCQQSHEYPFTFGQGTKLEIK

SEQ ID NO: 13 - protein, 8 aa, synthetic construct
  REGION 1..8: note = Synthetic: SD-633416_hCDR1
GYTFTSYD

SEQ ID NO: 14 - protein, 8 aa, synthetic construct
  REGION 1..8: note = Synthetic: SD-633416_hCDR2
IFPGDGTS

SEQ ID NO: 15 - protein, 7 aa, synthetic construct
  REGION 1..7: note = Synthetic: SD-633416_hCDR3
ARRLGSH

SEQ ID NO: 16 - protein, 10 aa, synthetic construct
  REGION 1..10: note = Synthetic: SD-633416_lCDR1
QSVDSDGDSY

SEQ ID NO: 17 - skipped

SEQ ID NO: 18 - protein, 9 aa, synthetic construct
  REGION 1..9: note = Synthetic: SD-633416_lCDR3
QQSHEYPFT

SEQ ID NO: 19 - DNA, 342 bp, synthetic construct
  misc_feature 1..342: note = Synthetic: SD-633416_VH_DNA
  source 1..342: mol_type = other DNA
gaagtgcaactggtgcaaagcggtgccgaagtcaagaagcccggagcctcagtgaaagtgtcctgcaaggcttcgggctacaccttcacctcctacgacattaactgggtcagacaggcacctggacagggcctggagtggatgggctggatcttcccgggcgacggaacttcgaaatacgcccagaagtttcagggtcgcgtgactatgactcgggatacttccacctccaccgtgtacatggaactcagctcccttcggtccgaggacaccgccgtctactattgtgcgaggagactggggtcacactggggacaggggacgaccgtgaccgtgtcgagc

SEQ ID NO: 20 - DNA, 333 bp, synthetic construct
  misc_feature 1..333: note = Synthetic: SD-633416_VL_DNA
  source 1..333: mol_type = other DNA
gatattcagatgacgcagagcccctcgtccctctccgcttccgtgggagatcgcgtcaccattacttgcaaagccagccagtccgtggactcggacggagactcctacatgaactggtaccagcagaagccaggaaaggccccgaagctgcttatctacggggcctccaacttggaatcgggagtgccttcacggttctctggttccggctccggcactgactttaccctgaccatcagcagcctgcagccggaggacttcgcgacttactactgccaacagtcacacgaatatcccttcaccttcggccaagggaccaagctggagatcaag

SEQ ID NO: 21 - protein, 114 aa, synthetic construct
  REGION 1..114: note = Synthetic: SD-382591_VH
EVQLVQSGAEVKKPGASVKVSCKASGYTFTSYDINWVRQAPGQGLEWMGWIFPGDGTSKYAQKFQGRVTMTTDTSTSTAYMELSSLRSEDTAVYYCARRLGSHWGQGTTVTVSS

SEQ ID NO: 22 - protein, 111 aa, synthetic construct
  REGION 1..111: note = Synthetic: SD-382591_VL
DIQLTQSPSSLSASVGDRVTITCKASQSVDSDGDSYMNWYQQKPGKAPKLLIYGASNLESGVPSRFSGSGSGTDFTLTISSLQPEDFATYYCQQSHEYPFTFGQGTKLEIK

SEQ ID NO: 23 - protein, 8 aa, synthetic construct
  REGION 1..8: note = Synthetic: SD-382591_hCDR1
GYTFTSYD

SEQ ID NO: 24 - protein, 8 aa, synthetic construct
  REGION 1..8: note = Synthetic: SD-382591_hCDR2
IFPGDGTS

SEQ ID NO: 25 - protein, 7 aa, synthetic construct
  REGION 1..7: note = Synthetic: SD-382591_hCDR3
ARRLGSH

SEQ ID NO: 26 - protein, 10 aa, synthetic construct
  REGION 1..10: note = Synthetic: SD-382591_lCDR1
QSVDSDGDSY

SEQ ID NO: 27 - skipped

SEQ ID NO: 28 - protein, 9 aa, synthetic construct
  REGION 1..9: note = Synthetic: SD-382591_lCDR3
QQSHEYPFT

SEQ ID NO: 29 - DNA, 342 bp, synthetic construct
  misc_feature 1..342: note = Synthetic: SD-382591_VH_DNA
  source 1..342: mol_type = other DNA
gaagtgcagctggtgcagtcaggcgccgaggtcaagaagcccggagcaagcgtgaaagtgtcctgcaaggcctcagggtacactttcacctcctatgacatcaactgggtcagacaggctccgggacaagggctcgaatggatgggttggattttccctggcgacggcacatcgaaatacgcgcagaagtttcagggacgcgtgaccatgaccaccgacacgtccacttccactgcctacatggaactgagctcgctgcggtccgaggataccgccgtgtactactgtgcccggaggcttggcagccactggggtcaaggaaccaccgtgactgtgtcctcg

SEQ ID NO: 30 - DNA, 333 bp, synthetic construct
  misc_feature 1..333: note = Synthetic: SD-382591_VL_DNA
  source 1..333: mol_type = other DNA
gatattcagctgacgcagagcccctcgtccctctccgcttccgtgggagatcgcgtcaccattacttgcaaagccagccagtccgtggactcggacggagactcctacatgaactggtaccagcagaagccaggaaaggccccgaagctgcttatctacggggcctccaacttggaatcgggagtgccttcacggttctctggttccggctccggcactgactttaccctgaccatcagcagcctgcagccggaggacttcgcgacttactactgccaacagtcacacgaatatcccttcaccttcggccaagggaccaagctggagatcaag

SEQ ID NO: 31 - protein, 114 aa, synthetic construct; identical to SEQ ID NO: 21
  REGION 1..114: note = Synthetic: SD-741396_VH

SEQ ID NO: 32 - protein, 111 aa, synthetic construct
  REGION 1..111: note = Synthetic: SD-741396_VL
DIQLTQSPSSLSASVGDRATITCKASQSVDSDGDSYMNWYQQKPGKAPKLLIYGASNLESGIPSRFSGSGSGTDFTLTISSVQPEDFATYYCQQSHEYPFTFGQGTKLEIK

SEQ ID NO: 33 - protein, 8 aa, synthetic construct
  REGION 1..8: note = Synthetic: SD-741396_hCDR1
GYTFTSYD

SEQ ID NO: 34 - protein, 8 aa, synthetic construct
  REGION 1..8: note = Synthetic: SD-741396_hCDR2
IFPGDGTS

SEQ ID NO: 35 - protein, 7 aa, synthetic construct
  REGION 1..7: note = Synthetic: SD-741396_hCDR3
ARRLGSH

SEQ ID NO: 36 - protein, 10 aa, synthetic construct
  REGION 1..10: note = Synthetic: SD-741396_lCDR1
QSVDSDGDSY

SEQ ID NO: 37 - skipped

SEQ ID NO: 38 - protein, 9 aa, synthetic construct
  REGION 1..9: note = Synthetic: SD-741396_lCDR3
QQSHEYPFT

SEQ ID NO: 39 - DNA, 342 bp, synthetic construct; identical to SEQ ID NO: 29
  misc_feature 1..342: note = Synthetic: SD-741396_VH_DNA
  source 1..342: mol_type = other DNA

SEQ ID NO: 40 - DNA, 333 bp, synthetic construct
  misc_feature 1..333: note = Synthetic: SD-741396_VL_DNA
  source 1..333: mol_type = other DNA
gatattcagttgacccagtccccgagctcactgtccgcttccgtgggtgatcgcgccactatcacgtgtaaagcgtcccagagcgtcgactccgacggggactcctacatgaactggtatcagcagaagcccggaaaggcccctaagctcctgatctacggcgcatccaacctggaaagcggaatcccctcgcggttctcgggaagcggctctgggaccgacttcacccttactatctcatcggtgcaaccggaggacttcgccacctactactgccaacagtcccacgaatacccattcacctttggacaaggcactaagctggagattaag

SEQ ID NO: 41 - protein, 114 aa, synthetic construct
  REGION 1..114: note = Synthetic: SD-844257_VH
EVQLVQSGAEVKKPGASVKLSCKASGYTFTSYDINWVRQAPGQGLEWIGWIFPGDGTSKYAQKFQGRATLTTDTSTSTAYMELSSLRSEDTAVYYCARRLGSHWGQGTTLTVSS

SEQ ID NO: 42 - protein, 111 aa, synthetic construct; identical to SEQ ID NO: 22
  REGION 1..111: note = Synthetic: SD-844257_VL

SEQ ID NO: 43 - protein, 8 aa, synthetic construct
  REGION 1..8: note = Synthetic: SD-844257_hCDR1
GYTFTSYD

SEQ ID NO: 44 - protein, 8 aa, synthetic construct
  REGION 1..8: note = Synthetic: SD-844257_hCDR2
IFPGDGTS

SEQ ID NO: 45 - protein, 7 aa, synthetic construct
  REGION 1..7: note = Synthetic: SD-844257_hCDR3
ARRLGSH

SEQ ID NO: 46 - protein, 10 aa, synthetic construct
  REGION 1..10: note = Synthetic: SD-844257_lCDR1
QSVDSDGDSY

SEQ ID NO: 47 - skipped

SEQ ID NO: 48 - protein, 9 aa, synthetic construct
  REGION 1..9: note = Synthetic: SD-844257_lCDR3
QQSHEYPFT

SEQ ID NO: 49 - DNA, 342 bp, synthetic construct
  misc_feature 1..342: note = Synthetic: SD-844257_VH_DNA
  source 1..342: mol_type = other DNA
gaagtgcagctcgtgcagtccggagccgaagtcaagaagccgggagcctcagtgaagctcagctgcaaggcctcgggctacaccttcacttcctacgacattaactgggtcagacaggcacctggacaaggcttggagtggatcggatggatctttcccggcgatgggactagcaaatacgcccagaagttccagggtcgcgcgactctgaccaccgacacctccacctcaaccgcgtatatggaactgtcctcccttcggtcggaggacactgccgtgtactactgcgctagaaggctgggcagccactggggtcaagggaccacactgacggtgtcgtcc

SEQ ID NO: 50 - DNA, 333 bp, synthetic construct; identical to SEQ ID NO: 30
  misc_feature 1..333: note = Synthetic: SD-844257_VL_DNA
  source 1..333: mol_type = other DNA

SEQ ID NO: 51 - protein, 114 aa, synthetic construct; identical to SEQ ID NO: 41
  REGION 1..114: note = Synthetic: SD-757052_VH

SEQ ID NO: 52 - protein, 111 aa, synthetic construct; identical to SEQ ID NO: 32
  REGION 1..111: note = Synthetic: SD-757052_VL

SEQ ID NO: 53 - protein, 8 aa, synthetic construct
  REGION 1..8: note = Synthetic: SD-757052_hCDR1
GYTFTSYD

SEQ ID NO: 54 - protein, 8 aa, synthetic construct
  REGION 1..8: note = Synthetic: SD-757052_hCDR2
IFPGDGTS

SEQ ID NO: 55 - protein, 7 aa, synthetic construct
  REGION 1..7: note = Synthetic: SD-757052_hCDR3
ARRLGSH

SEQ ID NO: 56 - protein, 10 aa, synthetic construct
  REGION 1..10: note = Synthetic: SD-757052_lCDR1
QSVDSDGDSY

SEQ ID NO: 57 - skipped

SEQ ID NO: 58 - protein, 9 aa, synthetic construct
  REGION 1..9: note = Synthetic: SD-757052_lCDR3
QQSHEYPFT

SEQ ID NO: 59 - DNA, 342 bp, synthetic construct; identical to SEQ ID NO: 49
  misc_feature 1..342: note = Synthetic: SD-757052_VH_DNA
  source 1..342: mol_type = other DNA

SEQ ID NO: 60 - DNA, 333 bp, synthetic construct; identical to SEQ ID NO: 40
  misc_feature 1..333: note = Synthetic: SD-757052_VL_DNA
  source 1..333: mol_type = other DNA

SEQ ID NO: 61 - protein, 114 aa, synthetic construct
  REGION 1..114: note = Synthetic: SD-787077_VH
EVQLVQSGAEVKKPGASVKVSCKASGYTFTSYDINWVRQAPGQGLEWMGWIFPGDGTSKYAQKFQGRVTMTTDKSTSTAYMELSSLRSEDTAVYYCARRLGSHWGQGTTVTVSS

SEQ ID NO: 62 - protein, 111 aa, synthetic construct; identical to SEQ ID NO: 22
  REGION 1..111: note = Synthetic: SD-787077_VL

SEQ ID NO: 63 - protein, 8 aa, synthetic construct
  REGION 1..8: note = Synthetic: SD-787077_hCDR1
GYTFTSYD

SEQ ID NO: 64 - protein, 8 aa, synthetic construct
  REGION 1..8: note = Synthetic: SD-787077_hCDR2
IFPGDGTS

SEQ ID NO: 65 - protein, 7 aa, synthetic construct
  REGION 1..7: note = Synthetic: SD-787077_hCDR3
ARRLGSH

SEQ ID NO: 66 - protein, 10 aa, synthetic construct
  REGION 1..10: note = Synthetic: SD-787077_lCDR1
QSVDSDGDSY

SEQ ID NO: 67 - skipped

SEQ ID NO: 68 - protein, 9 aa, synthetic construct
  REGION 1..9: note = Synthetic: SD-787077_lCDR3
QQSHEYPFT

SEQ ID NO: 69 - DNA, 342 bp, synthetic construct
  misc_feature 1..342: note = Synthetic: SD-787077_VH_DNA
  source 1..342: mol_type = other DNA
gaagtgcagctcgtgcaatccggcgccgaagtcaagaagcctggggcctcagtgaaggtgtcctgcaaagcatcggggtacaccttcacgagctacgacatcaactgggtccgccaagctccgggacagggtctggagtggatgggctggatttttcccggcgacggtaccagcaaatacgcgcagaagttccagggcagagtgaccatgaccaccgacaagtccacttcaaccgcctacatggagctgtcctccctgcggtcggaggatactgccgtgtattactgtgcccggaggcttggaagccactggggacagggaactactgtgaccgtgtcgtcc

SEQ ID NO: 70 - DNA, 333 bp, synthetic construct; identical to SEQ ID NO: 30
  misc_feature 1..333: note = Synthetic: SD-787077_VL_DNA
  source 1..333: mol_type = other DNA

SEQ ID NO: 71 - protein, 114 aa, synthetic construct; identical to SEQ ID NO: 61
  REGION 1..114: note = Synthetic: SD-638526_VH

SEQ ID NO: 72 - protein, 111 aa, synthetic construct; identical to SEQ ID NO: 32
  REGION 1..111: note = Synthetic: SD-638526_VL

SEQ ID NO: 73 - protein, 8 aa, synthetic construct
  REGION 1..8: note = Synthetic: SD-638526_hCDR1
GYTFTSYD

SEQ ID NO: 74 - protein, 8 aa, synthetic construct
  REGION 1..8: note = Synthetic: SD-638526_hCDR2
IFPGDGTS

SEQ ID NO: 75 - protein, 7 aa, synthetic construct
  REGION 1..7: note = Synthetic: SD-638526_hCDR3
ARRLGSH

SEQ ID NO: 76 - protein, 10 aa, synthetic construct
  REGION 1..10: note = Synthetic: SD-638526_lCDR1
QSVDSDGDSY

SEQ ID NO: 77 - skipped

SEQ ID NO: 78 - protein, 9 aa, synthetic construct
  REGION 1..9: note = Synthetic: SD-638526_lCDR3
QQSHEYPFT

SEQ ID NO: 79 - DNA, 342 bp, synthetic construct; identical to SEQ ID NO: 69
  misc_feature 1..342: note = Synthetic: SD-638526_VH_DNA
  source 1..342: mol_type = other DNA

SEQ ID NO: 80 - DNA, 333 bp, synthetic construct; identical to SEQ ID NO: 40
  misc_feature 1..333: note = Synthetic: SD-638526_VL_DNA
  source 1..333: mol_type = other DNA

SEQ ID NO: 81 - protein, 114 aa, synthetic construct
  REGION 1..114: note = Synthetic: SD-710726_VH
EVQLVQSGAEVKKPGASVKLSCKASGYTFTSYDINWVRQAPGQGLEWIGWIFPGDGTSKYAQKFQGRATLTTDKSTSTAYMELSSLRSEDTAVYYCARRLGSHWGQGTTLTVSS

SEQ ID NO: 82 - protein, 111 aa, synthetic construct; identical to SEQ ID NO: 22
  REGION 1..111: note = Synthetic: SD-710726_VL

SEQ ID NO: 83 - protein, 8 aa, synthetic construct
  REGION 1..8: note = Synthetic: SD-710726_hCDR1
GYTFTSYD

SEQ ID NO: 84 - protein, 8 aa, synthetic construct
  REGION 1..8: note = Synthetic: SD-710726_hCDR2
IFPGDGTS

SEQ ID NO: 85 - protein, 7 aa, synthetic construct
  REGION 1..7: note = Synthetic: SD-710726_hCDR3
ARRLGSH

SEQ ID NO: 86 - protein, 10 aa, synthetic construct
  REGION 1..10: note = Synthetic: SD-710726_lCDR1
QSVDSDGDSY

SEQ ID NO: 87 - skipped

SEQ ID NO: 88 - protein, 9 aa, synthetic construct
  REGION 1..9: note = Synthetic: SD-710726_lCDR3
QQSHEYPFT

SEQ ID NO: 89 - DNA, 342 bp, synthetic construct
  misc_feature 1..342: note = Synthetic: SD-710726_VH_DNA
  source 1..342: mol_type = other DNA
gaagtgcagttggtgcagtcgggagccgaagtcaagaagcctggagcgtccgtgaagctgagctgcaaggcctcaggatacactttcacttcatatgacatcaactgggtcagacaggcaccgggccaaggactggagtggattggctggatctttcccggggatggcacgagcaaatacgcccagaagttccagggtagagcgaccctgaccaccgacaagtccacttcgaccgcctacatggaactctcctcgctgcgctccgaggacaccgccgtgtactactgtgctcggaggcttgggtcccactggggtcaaggcaccactctcaccgtgtccagc

SEQ ID NO: 90 - DNA, 333 bp, synthetic construct; identical to SEQ ID NO: 30
  misc_feature 1..333: note = Synthetic: SD-710726_VL_DNA
  source 1..333: mol_type = other DNA

SEQ ID NO: 91 - protein, 114 aa, synthetic construct; identical to SEQ ID NO: 81
  REGION 1..114: note = Synthetic: SD-577776_VH

SEQ ID NO: 92 - protein, 111 aa, synthetic construct; identical to SEQ ID NO: 32
  REGION 1..111: note = Synthetic: SD-577776_VL

SEQ ID NO: 93 - protein, 8 aa, synthetic construct
  REGION 1..8: note = Synthetic: SD-577776_hCDR1
GYTFTSYD

SEQ ID NO: 94 - protein, 8 aa, synthetic construct
  REGION 1..8: note = Synthetic: SD-577776_hCDR2
IFPGDGTS

SEQ ID NO: 95 - protein, 7 aa, synthetic construct
  REGION 1..7: note = Synthetic: SD-577776_hCDR3
ARRLGSH

SEQ ID NO: 96 - protein, 10 aa, synthetic construct
  REGION 1..10: note = Synthetic: SD-577776_lCDR1
QSVDSDGDSY

SEQ ID NO: 97 - skipped

SEQ ID NO: 98 - protein, 9 aa, synthetic construct
  REGION 1..9: note = Synthetic: SD-577776_lCDR3
QQSHEYPFT

SEQ ID NO: 99 - DNA, 342 bp, synthetic construct; identical to SEQ ID NO: 89
  misc_feature 1..342: note = Synthetic: SD-577776_VH_DNA
  source 1..342: mol_type = other DNA

SEQ ID NO: 100 - DNA, 333 bp, synthetic construct; identical to SEQ ID NO: 40
  misc_feature 1..333: note = Synthetic: SD-577776_VL_DNA
  source 1..333: mol_type = other DNA

SEQ ID NO: 101 - protein, 114 aa, synthetic construct
  REGION 1..114: note = Synthetic: SD-127612_VH
QVQLVQSGAEVKKPGASVKVSCKASGYTFTSYDINWVRQAPGQRLEWMGWIFPGDGTSKYNEKFKGRVTITRDTSASTAYMELSSLRSEDTAVYYCARRLGSHWGQGTLVTVSS

SEQ ID NO: 102 - protein, 111 aa, synthetic construct
  REGION 1..111: note = Synthetic: SD-127612_VL
DIVLTQSPDSLAVSPGERATISCKASQSVDSDGDSYMNWYQQKPGQPPKLLIYGASNLESGVPDRFSGSGSGTDFTLTISRVEAEDVAVYYCQQSHEYPFTFGGGTKLEIK

SEQ ID NO: 103 - protein, 8 aa, synthetic construct
  REGION 1..8: note = Synthetic: SD-127612_hCDR1
GYTFTSYD

SEQ ID NO: 104 - protein, 8 aa, synthetic construct
  REGION 1..8: note = Synthetic: SD-127612_hCDR2
IFPGDGTS

SEQ ID NO: 105 - protein, 7 aa, synthetic construct
  REGION 1..7: note = Synthetic: SD-127612_hCDR3
ARRLGSH

SEQ ID NO: 106 - protein, 10 aa, synthetic construct
  REGION 1..10: note = Synthetic: SD-127612_lCDR1
QSVDSDGDSY

SEQ ID NO: 107 - skipped

SEQ ID NO: 108 - protein, 9 aa, synthetic construct
  REGION 1..9: note = Synthetic: SD-127612_lCDR3
QQSHEYPFT

SEQ ID NO: 109 - DNA, 342 bp, synthetic construct
  misc_feature 1..342: note = Synthetic: SD-127612_VH_DNA
  source 1..342: mol_type = other DNA
caagttcagctcgtgcagagtggagcagaagtgaaaaagccaggggcttcagttaaggtaagctgtaaagcctccgggtatacattcacatcatacgacataaattgggtgaggcaagccccgggtcagcggctggagtggatggggtggattttccccggcgatgggacttccaagtacaatgaaaagttcaaggggcgagtgacaatcactagggacacttccgccagcacggcttacatggaactcagctcactcagaagtgaggataccgcggtctattactgtgctcgcaggctgggatcccactggggccaagggactctggttacagtctcctcc

SEQ ID NO: 110 - DNA, 333 bp, synthetic construct
  misc_feature 1..333: note = Synthetic: SD-127612_VL_DNA
  source 1..333: mol_type = other DNA
gacattgtactcacccagagtccagacagcttggccgtcagtccaggtgagagagccaccattagctgcaaggcatctcagagcgtggatagtgatggcgatagctacatgaactggtaccagcagaaaccaggccagccacctaagctcctcatctatggcgcctctaaccttgaatctggagtgcccgaccgctttagcggtagcggcagcggcacagatttcactttgacaattagtcgcgtggaggccgaagacgtggcagtctactactgccagcagagccacgagtacccattcacattcgggggagggacaaagttagagattaag

SEQ ID NO: 111 - protein, 114 aa, synthetic construct; identical to SEQ ID NO: 101
  REGION 1..114: note = Synthetic: SD-837152_VH

SEQ ID NO: 112 - protein, 111 aa, synthetic construct
  REGION 1..111: note = Synthetic: SD-837152_VL
DIVLTQSPASLAVSPGQRATITCKASQSVDSDGDSYMNWYQQKPGQPPKLLIYGASNLESGVPARFSGSGSGTDFTLTINPVEANDTANYYCQQSHEYPFTFGQGTKLEIK

SEQ ID NO: 113 - protein, 8 aa, synthetic construct
  REGION 1..8: note = Synthetic: SD-837152_hCDR1
GYTFTSYD

SEQ ID NO: 114 - protein, 8 aa, synthetic construct
  REGION 1..8: note = Synthetic: SD-837152_hCDR2
IFPGDGTS

SEQ ID NO: 115 - protein, 7 aa, synthetic construct
  REGION 1..7: note = Synthetic: SD-837152_hCDR3
ARRLGSH

SEQ ID NO: 116 - protein, 10 aa, synthetic construct
  REGION 1..10: note = Synthetic: SD-837152_lCDR1
QSVDSDGDSY

SEQ ID NO: 117 - skipped

SEQ ID NO: 118 - protein, 9 aa, synthetic construct
  REGION 1..9: note = Synthetic: SD-837152_lCDR3
QQSHEYPFT

SEQ ID NO: 119 - DNA, 342 bp, synthetic construct; identical to SEQ ID NO: 109
  misc_feature 1..342: note = Synthetic: SD-837152_VH_DNA
  source 1..342: mol_type = other DNA

SEQ ID NO: 120 - DNA, 333 bp, synthetic construct
  misc_feature 1..333: note = Synthetic: SD-837152_VL_DNA
  source 1..333: mol_type = other DNA
gatatcgttctgacccagagtccagcgtccttggccgtgtctccaggccaacgggctacaataacatgcaaggcctctcagagcgtggactctgacggcgatagttacatgaattggtatcagcagaagcctggacagccccccaaactgttgatttacggggcaagcaacctggagagtggcgtgcccgcacgtttttcaggctcagggagcggtaccgactttacacttacaattaatcccgtggaggctaatgatactgccaactactactgtcagcagagccacgagtatccttttacttttggccagggaaccaaactggaaatcaaa

SEQ ID NO: 121 - protein, 114 aa, synthetic construct
  REGION 1..114: note = Synthetic: SD-649072_VH
QVQLVQSGAEVKKPGASVKVSCKASGYTFTSYDINWVRQAPGQRLEWMGWIFPGDGTSKYNEKFKGRVTITRDTSASTAYMELSSLRSEDTAVYYCARRLGTVWGQGTLVTVSS

SEQ ID NO: 122 - protein, 111 aa, synthetic construct; identical to SEQ ID NO: 112
  REGION 1..111: note = Synthetic: SD-649072_VL

SEQ ID NO: 123 - protein, 8 aa, synthetic construct
  REGION 1..8: note = Synthetic: SD-649072_hCDR1
GYTFTSYD

SEQ ID NO: 124 - protein, 8 aa, synthetic construct
  REGION 1..8: note = Synthetic: SD-649072_hCDR2
IFPGDGTS

SEQ ID NO: 125 - protein, 7 aa, synthetic construct
  REGION 1..7: note = Synthetic: SD-649072_hCDR3
ARRLGTV

SEQ ID NO: 126 - protein, 10 aa, synthetic construct
  REGION 1..10: note = Synthetic: SD-649072_lCDR1
QSVDSDGDSY

SEQ ID NO: 127 - skipped

SEQ ID NO: 128 - protein, 9 aa, synthetic construct
  REGION 1..9: note = Synthetic: SD-649072_lCDR3
QQSHEYPFT

SEQ ID NO: 129 - DNA, 342 bp, synthetic construct
  misc_feature 1..342: note = Synthetic: SD-649072_VH_DNA
  source 1..342: mol_type = other DNA
caagttcagctcgtgcagagtggagcagaagtgaaaaagccaggggcttcagttaaggtaagctgtaaagcctccgggtatacattcacatcatacgacataaattgggtgaggcaagccccgggtcagcggctggagtggatggggtggattttccccggcgatgggacttccaagtacaatgaaaagttcaaggggcgagtgacaatcactagggacacttccgccagcacggcttacatggaactcagctcactcagaagtgaggataccgcggtctattactgtgctcgcaggctgggaaccgtttggggccaagggactctggttacagtctcctcc

SEQ ID NO: 130 - DNA, 333 bp, synthetic construct; identical to SEQ ID NO: 120
  misc_feature 1..333: note = Synthetic: SD-649072_VL_DNA
  source 1..333: mol_type = other DNA

SEQ ID NO: 131 - protein, 15 aa, synthetic construct
  REGION 1..15: note = Synthetic: linker
GGGGSGGGGSGGGGS

SEQ ID NO: 132 - protein, 19 aa, synthetic construct
  REGION 1..19: note = Synthetic: EGFRvIII
LEEKKGNYVVTDHGSGSGC

SEQ ID NO: 133 - protein, 23 aa, synthetic construct
  REGION 1..23: note = Synthetic: EGFRvIII
GADSYEMEEDGVRKSKKTEGSGC

SEQ ID NO: 134 - protein, 41 aa, synthetic construct
  REGION 1..41: note = Synthetic: EGFRvIII
LEEKKGNYVVTDHGSSVRADGADSYEMEEDGVRKTKKGSGC

(19) United States
(12) Patent Application Publication
(10) Pub. No.: US 2024/0117056 A1
(43) Pub. Date: Apr. 11, 2024

(54) EPIDERMAL GROWTH FACTOR RECEPTOR VARIANT III ANTIBODIES

(71) Applicant: iBio, Inc., Bryan, TX (US)

(72) Inventors: Dillon Phan, La Jolla, CA (US); Tom Sih-Yuan Hsu, Chino, CA (US); Matthew P. Greving, Rancho Santa Fe, CA (US); Martin Brenner, San Diego, CA (US); Tam Thi Thanh Phuong, San Diego, CA (US); Alexander Tomoaki Taguchi, San Diego, CA (US); Cory Schwartz, San Diego, CA (US); Gao Liu, San Diego, CA (US); Jiang Chen, San Diego, CA (US)

(21) Appl. No.: 18/484,635

(22) Filed: Oct. 11, 2023

Related U.S. Application Data
(60) Provisional application No. 63/415,051, filed on Oct. 11, 2022.
(60) Provisional application No. 63/515,366, filed on Jul. 25, 2023.

(51) Int. Cl. C07K 16/28; A61P 35/00

(52) U.S. Cl. CPC C07K 16/2863; A61P 35/00; A61K 2039/505

(57) ABSTRACT

Provided herein are anti-EGFRvII antibodies and binding fragments thereof. The anti-EGFRvIII antibodies of the disclosure are useful for the treatment of cancers through, e.g., antibody-dependent cell cytotoxicity (ADCC). Also provided herein are methods of making and using the anti-EGFRvIII antibodies for the treatment of cancer, and polynucleotides that encode the same.

### CROSS-REFERENCE TO RELATED APPLICATIONS

This application claims priority to U.S. Provisional Application Ser. No. 63/415,051, filed Oct. 11, 2022, and 63/515,366, filed Jul. 25, 2023, and, the entire contents of which are incorporated herein by reference.

### TECHNICAL FIELD OF THE INVENTION

This document relates to materials and methods for treating cancer, and particularly to the use of anti-EGFRvIII antibodies to reduce or eliminate cells that express the truncated EGFRvIII, and to treat cancer.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Oct. 10, 2023, is named “IBIO1038.xml” and is 166,298 bytes in size. The sequence listing contained in this IBIO1038.XML file is part of the specification and is hereby incorporated by reference herein in its entirety.

### BACKGROUND OF THE INVENTION

Without limiting the scope of the invention, its background is described in connection with cancers that express anti-EGFRvIII.

Epidermal growth factor receptor (EGFR1/ErbB1/HER1) is a member of a tyrosine receptor family and activated by the ligand EGF. EGFR1 overexpression is commonly found in many cancer types. EGFR1 is found to be mutated in certain tumors, with most common mutation being the EGFR variant III (EGFRvIII). EGFRvIII has a unique in-frame deletion of 267 aa from exon 2 to 7 in the ECD of EGFR, leading to the inability to bind EGF ligand. EGFRvIII expression leads to resistance to conventional EGFR1-targeting therapies.

Although frequency of EGFRvIII expression in tumors changes based on the tumor type, EGFRvIII expression is specific to tumor cells only. Thus, EGFRvIII is a desirable therapeutic target due to its specific expression in tumor cells. The EGFRvIII accounts for 30-40% of glioblastomas, 8-42% of head and neck squamous cell carcinomas, 3-16% of Non-Small Cell Lung Cancer-Squamous Cell Carcinoma (NSCLC-SCC), up to 6.5% of prostate cancers, up to 27% of breast cancers, and up to 8% of colon cancers.

What is needed are novel antibodies that bind specifically to EGFRvIII without binding to wild-type EGFR. Also needed are novel antibodies that enhance antibody-dependent cell cytotoxicity.

### SUMMARY OF THE INVENTION

As embodied and broadly described herein, an aspect of the present disclosure relates to an anti-Epidermal Growth Factor Receptor version III (EGFRvIII) antibody or antigen binding domain thereof, wherein the antibody or antigen binding domain comprises: a heavy chain variable domain (VH) complementarity determining region (CDR) 1, VH CDR2 and VH CDR3 comprising an amino acid sequence of any one of the following SEQ ID NOs: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; or 123, 124, 125, respectively; and a light chain variable domain (VL) CDR1, VL CDR2 and VL CDR3 comprising an amino acid sequence of any one of the following SEQ ID NOs: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; or 126, 127, 128, respectively. In one aspect, the antibody comprises a VH comprising the amino acid sequence of any one of the following SEQ ID NOs: 1, 11, 21, 31, 41, 51, 61, 71, 81, 91, 101, 111, or 121. In another aspect, the antibody comprises a VL comprising the amino acid sequence of any one of the following SEQ ID NOs: 2, 12, 22, 32, 42, 52, 62, 72, 82, 92, 102, 112, or 122. In another aspect, the antibody is a monoclonal antibody. In another aspect, the antibody is a full-length antibody. In another aspect, the antibody is an antibody fragment. In another aspect, the antibody is fused to an Fc domain of any one of the following: human IgG1, human IgG2, human IgG3, and human IgG4. In another aspect, the antibody heavy chain comprises an amino acid sequence with at least 80%, 85%, 90%, 95, 96, 97, 98, 99% or 100% sequence identity with SEQ ID NOS: 1, 11, 21, 31, 41, 51, 61, 71, 81, 91, 101, 111, or 121, or antibodies that comprises therewith. In another aspect, the antibody light chain comprises as amino acid sequence with at least 80%, 85%, 90%, 95, 96, 97, 98, 99% or 100% sequence identity with SEQ ID NOS: 2, 12, 22, 32, 42, 52, 62, 72, 82, 92, 102, 112, or 122. In another aspect, the antibody comprises a heavy chain variable domain and light chain variable domain: SEQ ID NO: 1 and 2, 11 and 12, 21 and 22, 31 and 32, 41 and 42, 51 and 52, 61 and 62, 71 and 72, 81 and 82, 91 and 92, 101 and 102, 111 and 112, or 121 and 122, respectively. In another aspect, the antibody heavy chain is encoded by a nucleic acid and the antibody light chain is encoded by a nucleic acid with at least 80%, 85%, 90%, 95, 96, 97, 98, 99% or 100% sequence identity to SEQ ID NOS: 9 and 10, 19 and 20, 29 and 30, 39 and 40, 49 and 50, 59 and 60, 69 and 70, 79 and 80, 89 and 90, 99 and 100, 109 and 110, 119 and 120, or 129 and 130. In another aspect, the antibody or binding domain is afucosylated. In another aspect, the antibody or binding domain is produced in a bacteria, fungal, mammalian, insect, or plant cell. As embodied and broadly described herein, an aspect of the present disclosure relates to a method of treating a disease in a subject in need thereof, comprising administering to the subject a therapeutically effective amount of the antibody described herein. In one aspect, the disease is a cancer. In another aspect, the disease is cancer selected from glioblastoma, head and neck squamous cell carcinoma, Non-Small Cell Lung Cancer-Squamous Cell Carcinoma (NSCLC-SCC), prostate cancer, breast cancer, and colon cancer, wherein the cancer expresses EGFRvIII. In another aspect, the cancer cells of the cancer are killed by antibody-dependent cell cytotoxicity (ADCC). In another aspect, the subject is human. In another aspect, the antibody or binding fragment thereof does not bind EGFR1.

As embodied and broadly described herein, an aspect of the present disclosure relates to a polynucleotide that comprises a nucleic acid sequence with at least 80%, 85%, 90%, 95, 96, 97, 98, 99%, or 100% sequence identity with SEQ ID NOS: 9 and 10, 19 and 20, 29 and 30, 39 and 40, 49 and 50, 59 and 60, 69 and 70, 79 and 80, 89 and 90, 99 and 100, 109 and 110, 119 and 120, or 129 and 130, respectively. In another aspect, the subject is human. In another aspect, the antibody or binding fragment thereof does not bind EGFR1.

As embodied and broadly described herein, an aspect of the present disclosure relates to a vector comprising the polynucleotide described herein. As embodied and broadly described herein, an aspect of the present disclosure relates to a host cell comprising the vector described herein. As embodied and broadly described herein, an aspect of the present disclosure relates to a method of making an anti-EGFRvIII antibody comprising expressing in a cell a nucleic acid encoding an antibody described herein. In another aspect, the subject is human. In another aspect, the antibody or binding fragment thereof does not bind EGFR1.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C compare binding of the various antibodies by surface plasmon resonance binding to EGFRvIII and EGFR1, respectively (FIG. 1A, SD-127612-afuc, SD-233883-afuc, Cetuximab, SD-382591-afuc, hIgG1 negative control, SD-577776-afuc), (FIG. 1B, SD-633416-afuc, SD-638526-afuc, SD-649072-afuc, SD-710726-afuc, SD-741396-afuc, SD-757052-afuc), and (FIG. 1C, SD-787077-afuc, SD-837152-afuc, SD-844257-afuc).

FIGS. 2A to 2D compare binding by ELISA to human EGFRvIII and EGFR1 by the hIgG1 isotype control antibody, Cetuximab, and the chimera and humanized antibodies of the present invention.

FIGS. 3A to 3F compare binding by chimera and humanized anti-EGFRvIII antibodies of the present invention. The antibodies listed bind specifically to human EGFRvIII but not to wild type human EGFR1. FACS analysis shows anti-EGFRvIII antibodies binding specifically to F98 rat glioblastoma (FIG. 3A), U87MG human glioblastoma (FIG. 3C), and FaDu human head and neck cancer cells (FIG. 3E) that are overexpressing human EGFRvIII. No binding was detected to F98 cells overexpressing wild type human EGFR1 cells (FIG. 3B) or to wild type U87MG (FIG. 3D) and FaDu (FIG. 3F) cells. Values plotted are Median Fluorescent Intensity. EC₅₀ values are averages of n=3 experiments. n/a, no activity.

FIGS. 4A to 4F show chimera and humanized anti-EGFRvIII antibodies of the present invention demonstrating potent ADCC activity against F98 (FIG. 4A), U87MG (FIG. 4C), and FaDu cells (FIG. 4E) overexpressing human EGFRvIII but not against human EGFR1 expressing F98 (FIG. 4B), wild type U87MG (FIG. 4D) and FaDu (FIG. 4F) cells. The ratio of dead cells to the total cells was used to determine the percentage of cell lysis. EC₅₀ values are averages of n=1-5 experiments. n/a, no activity.

FIGS. 5A to 5E show: FIG. 5A study design for in vivo efficacy of anti-EGFRvIII antibody SD-233883-afuc against FaDu EGFRvIII tumor cells in a nude mice model. FIG. 5B (FaDu-EGFRvIII tumor volume after initial drug treatment) and FIG. 5C (FaDu-EGFRvIII tumor volume change % after initial drug treatment) show that SD-233883-afuc significantly inhibited FaDu-EGFRvIII tumor growth throughout the entire observation time window (T-test, P<0.05) in comparison with hIgG1 negative control. FIG. 5D shows at the endpoint (day 22) of the study, FaDu-EGFRvIII tumor weight was significantly reduced after the treatment of Cetuximab or SD-233883-afuc (T-test, P<0.05) in comparison with hIgG1 negative control. FIG. 5E shows mouse body weight after initial drug treatment.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

The epidermal growth factor receptor (EGFR1/ErbB1/HER1) is a member of a tyrosine receptor family and is activated by the ligand epidermal growth factor (EGF). EGFR1 overexpression is commonly found in many cancer types. EGFR1 is also found to be mutated in certain tumors, with most common mutation being the EGFR variant III (EGFRvIII). EGFRvIII has a unique in-frame deletion of 267 aa from exon 2 to 7 in the ECD of EGFR, leading to the inability to bind EGF ligand. Although the frequency of EGFRvIII expression in tumors changes based on the tumor type, EGFRvIII expression is specific to tumor cells only. For those cancers expressing EGFRvIII, this is a desirable therapeutic target due to its specific expression in tumor cells. Importantly, EGFRvIII expression leads to resistance to conventional EGFR1-targeting therapies.

The present invention are novel antibodies that are EGFRvIII-specific and that do not bind the native or wild-type EGFR1. Afucosylated antibodies lead to enhanced ADCC response and no binding to wild-type or native EGFR1, which provides a better safety profile.

The anti-EGFRvIII-specific ADCC approach allows for selective destruction of EGFRvIII expressing tumor cells and minimizing off-target effects.

As used herein, the term “meso scale-molecule (MEM)” refers to engineered peptides and polypeptides between about 1 kDa and about 10 kDa. The term “MEM-nanoparticle” as used herein throughout includes MEMs which have been conjugated to a nanoparticle (e.g., ferritin nanoparticle).

As used herein, a “subject” may be a mammalian or avian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

All publications, patents, and patent applications mentioned in this specification are herein incorporated by reference to the same extent as if each individual publication, patent, or patent application was specifically and individually indicated to be incorporated by reference.

Antibodies.

As used herein, the term “antibody” refers to an intact antibody or a binding fragment thereof that binds specifically to a target antigen, in the present invention, EGRFvIII. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including mammalian, yeast, and plant cells.

As used herein, the term “antibody fragment” refers to a portion of a full-length antibody, generally the antigen-binding or variable region, and include Fab, Fab′, F(ab′)₂, Fv, and scFv fragments. The antibody fragments or domains of the disclosure retain EGFRvIII antigen binding specificity. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequenator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the 3-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for EGFRvIII. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fᵥ framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component C1q binding and/or the complement-dependent cytotoxicity (CDC) function of the Fc region of an IL-23p19 binding molecule. The binding polypeptide of particular interest may be one that binds to C1q and displays complement-dependent cytotoxicity. Polypeptides with pre-existing C1q binding activity, optionally further having the ability to mediate CDC may be modified such that one or both of these activities are enhanced. Amino acid modifications that alter C1q and/or modify its complement-dependent cytotoxicity function are described, for example, in WO/0042072, which is hereby incorporated by reference.

An Fc region of an antibody can be designed to alter the effector function, e.g., by modifying C1q binding and/or FcTR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: C1q binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The antigen of the present invention is the EGFRvII, which also include MEMs of the same. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

### Example 1. EGFRvIII MEMs

The meso scale-molecules (MEMs) of the disclosure are made to mimic the agonist epitope identified in Table 1 and are subsequently used to screen for antibodies. The advantage of this approach is the ability to steer antibody discovery away from the wild-type receptor and toward the desired epitope that is only found in EGFRvIII. The scaffold amino acids are in bold, and the epitope residues are underlined.

TABLE 1
MEMs sequences used for immunization.

| | | SEQ ID |
|---|---|---|
| Target | Sequence (Scaffold, EGFRVIII epitope) | NO |
| EGFRvIII | LEEKKGNYVVTDHGSGSGC | 132 |
| EGFRvIII | GADSYEMEEDGVRKSKKTEGSGC | 133 |
| EGFRvIII | LEEKKGNYVVTDHGSSVRADGADSYEMEEDGVRKT | 134 |
| | KKGSGC | |

In some embodiments, the MEM-nanoparticles were used to immunize a subject in order to produce antibodies specific to the MEM epitope. Monoclonal hybridomas were then created to produce epitope specific anti-EGFRvIII antibodies. Humanized anti-EGFRvIII CDRs were determined based on reference antibodies.

### Example 2. Anti-EGFRvIII Antibodies Discovery Based on Engineered MEM-Nanoparticle Immunizations

MEMs were designed based on the epitope identified by SEQ ID NO: 47, 48, and 49, and then conjugated to nanoparticles to steer B-cell antibody production towards said epitopes. MEMs conjugated to ferritin nanoparticles were identified by Coomassie-based western blots and were found to contain approximately 20-30 MEMs per nanoparticle. The MEM-nanoparticles demonstrated nanomolar binding affinity to anti-EGFRvIII using Surface Plasmon Resonance (SPR). BALB/c mice were then immunized over a 5-week period with alternating doses of the engineered MEM-nanoparticle and/or full-length anti-EGFRvIII suspended in adjuvant, with a final boost containing a combination of the two. Mouse serum was collected, yielding strong anti-EGFRvIII binding measured via ELISA.

Top Monoclonal Hybridoma Produced Antibody Demonstrates Strong anti-EGFRvIII Binding and Agonism. Hybridomas were created from the immunized mouse B-cells using standard electro cell fusion methods. The resulting antibodies and several others were generated from monoclonal hybridomas and demonstrated strong anti-EGFRvIII binding via ELISA. Binding was further assessed in vitro for anti-EGFRvIII binding and competition binding for anti-EGFRvIII using SPR.

Antibody expression and purification. Antibody expression plasmids were transiently introduced into an animal cell line using the ExpiFectamine CHO Transfection Kit (Thermo Fisher, Cat #A29129) to yield transfectants that produced anti-CCR8 chimeric or humanized antibody. For a host cell line, ExpiCHO-S (Thermo Fisher, Cat #A29127) or a suspension CHO cell line with the α1,6 fucosyltransferase (FUT8) gene knocked out were used (referred to as “WT CHO” and “FUT8 CHO” in further references). After 6-12 days of growth post introduction of DNA, cell suspensions of WT CHO or FUT8 CHO were harvested via centrifugation for 20 minutes at 4,000×g, and then filtered using 0.2 μm Disposable PES Filter units (Fisher Scientific, Cat #FB12566504). Antibody was recovered from filtrate using Protein A purification (HiTrap MabSelect SuRe; Cytiva, Cat #GE11-0034-93). WT CHO was used to express antibodies with standard glycosylation, and FUT8 CHO used to express afucosylated antibodies with enhanced effector function (indicated by “-afuc”).

Antibody humanization. Humanization was accomplished via multiple approaches. In some instances, rationally selected framework amino acids that were different between the chimera (SD-233883) and the closest human germline were converted to match the human sequence. In other cases, a publicly available tool (DOI: 10.1080/19420862.2021.2020203) was used to graft the CDRs directly onto a human germline. In all cases, the CDRs were left unchanged. Humanized variants were then tested and potency compared to the parental chimera.

Preparation of phage display library. CDR variant libraries were prepared based on the parental antibody. VH and VL sequences were assembled with golden gate assembly method and ligated into digested phagemid vector for phage display as ScFvs. Ligations were transformed into Phage-Competent™ TG1 Cells (Antibody design labs, Cat #PC001), and library quality determined by size and VH/VL insert percentage.

Phage display screening. Synthetic library phage display selection was performed using soluble protein antigen. The selections were performed using biotinylated huEGFRvIII, with Dynabeads M-280 Streptavidin beads Magnetic Beads (Invitrogen; Cat #11205D) on a KingFisher Apex. Antigen was used at a range of concentrations. Elution was done with TEA (Triethylamine) (Sigma; Cat #T0886), and selection buffer was skimmed milk in 1×PBS. After 3 rounds of panning, the plasmids were extracted and the VH/VL genes amplified for analysis via Sanger sequencing. VH/VL sequences of interest were cloned into IgG1 expression plasmids and then transformed into DH5α. Each plasmid was extracted from DH5α, and then transfected into CHO cells in 24 deep well plates for expression using ExpiFectamine CHO Transfection Kit. The purification was done using KingFisher Apex. Phage matured clones were then screened using SPR.

### Example 3. Kinetic Analysis of Anti-EGFRvIII Antibodies by Surface Plasmon Resonance

Kinetic analysis of anti-EGFRvIII antibodies by surface plasmon resonance. Binding of antibodies to human EGFR and EGFRvIII was assessed by surface plasmon resonance (SPR) using the Carterra LSA (Carterra, Inc.). An anti-human IgG capture lawn was first prepared on an HC30M chip (Carterra, Cat #4279) by primary amine coupling. Briefly, the chip surface was activated for 10 minutes with a mixture of 133 mM EDC (Thermo Fisher, Cat #22980) and 33.3 mM sulfo-NHS (Thermo Fisher, Cat #24525) in 100 mM MES pH 5.5 (Carterra, Cat #3625), after which the goat anti-human IgG (Southern Biotech, Cat #2040-01) was coupled for 15 minutes at 50 μg/mL in 10 mM sodium acetate buffer at pH 4.5 (Carterra, Cat #3622). The unconjugated space on the chip surface was blocked with 1 M ethanolamine HCL pH 8.5 (Carterra, Cat #3626) for 7 minutes. For capture kinetics, the prepared anti-human IgG surface and the 96 channel printhead (96PH) was used to capture an antibody panel for 10 minutes at 1-10 μg/mL in HBSTE buffer (10 mM HEPES pH 7.4, 150 mM NaCl, 3 mM EDTA, 0.05% Tween 20; Carterra, Cat #3630). Purified recombinant antigens (human EGFR, Acro Biosystems Cat #EGR-H5222; human EGFRvIII, Cat #EGI-H52H4) were then injected using the single flow cell (SFC) over the antibody panel at 5 concentrations in a 5-fold dilution series, beginning at 500 nM. Each injection used a 5-minute association phase and a 15-minute dissociation phase. The surface was regenerated between antigens with 0.425% H3PO4 (Carterra, Cat #3637). The running buffer for antigen injections was HBSTE supplemented with 0.5 mg/mL BSA (VWR, Cat #97061-422). Binding data was double referenced by subtracting an interspot reference response as well as buffer-only blank responses. The resulting sensorgrams were globally fit to a 1:1 Langmuir binding model to estimate the association rate constant (ka), dissociation rate constant (kd), and the dissociation constant (KD) using the Carterra Kinetics software.

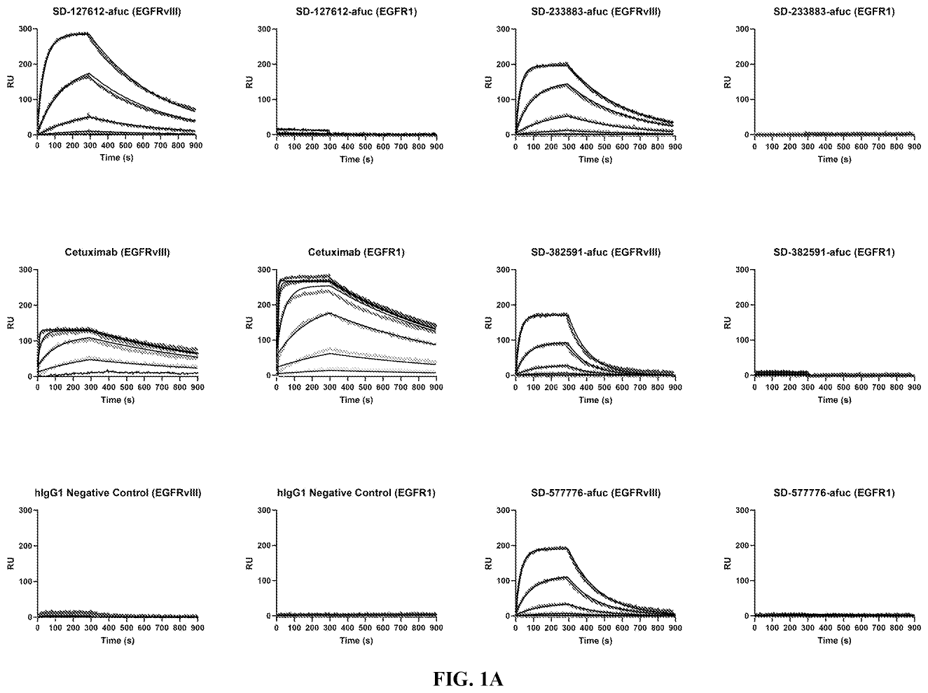

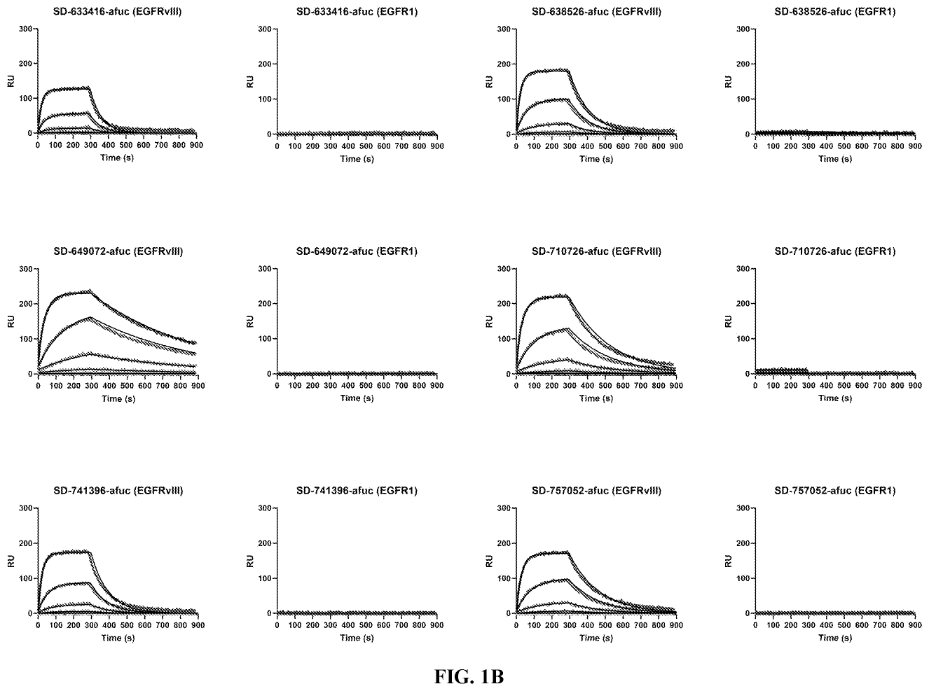

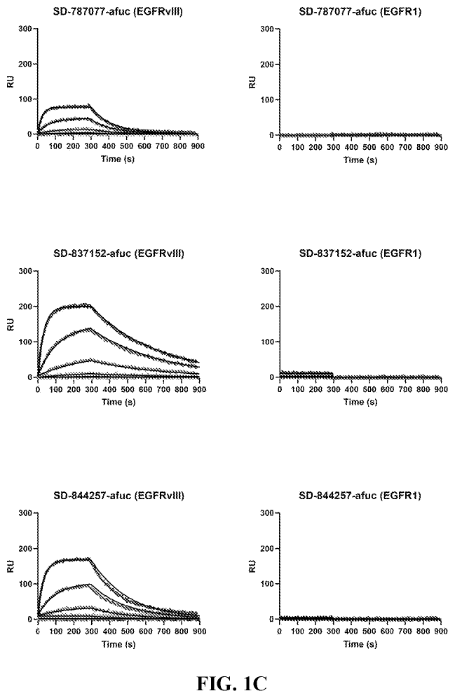

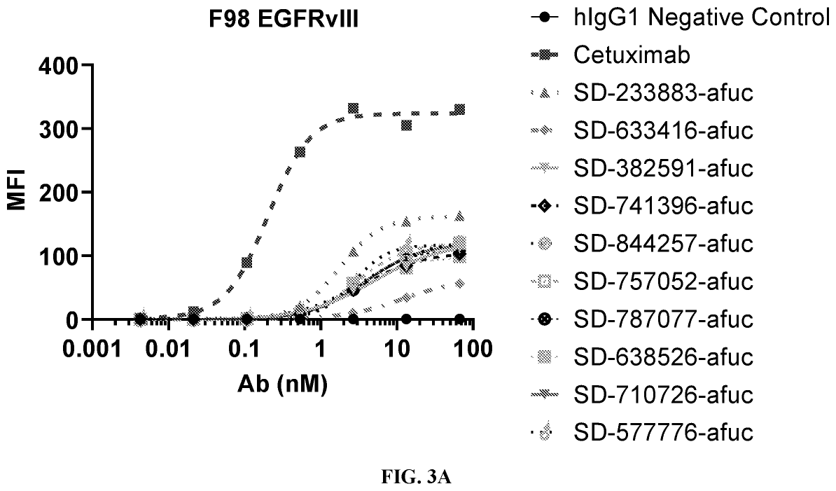

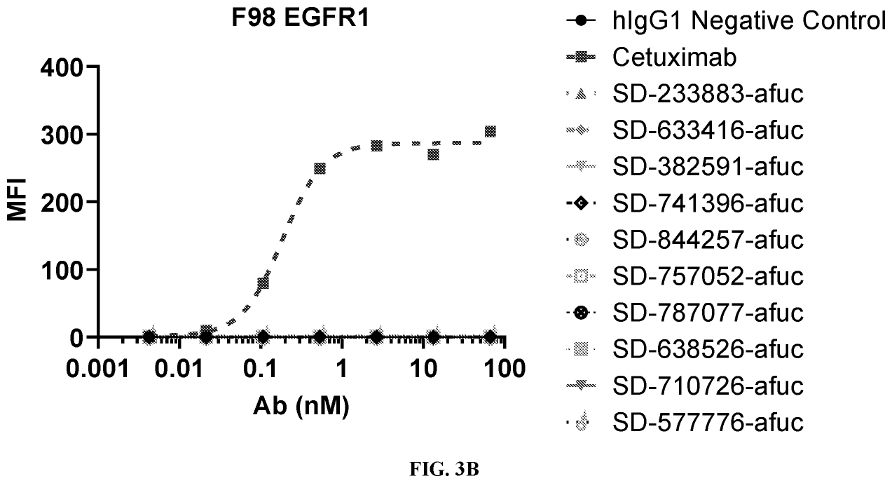

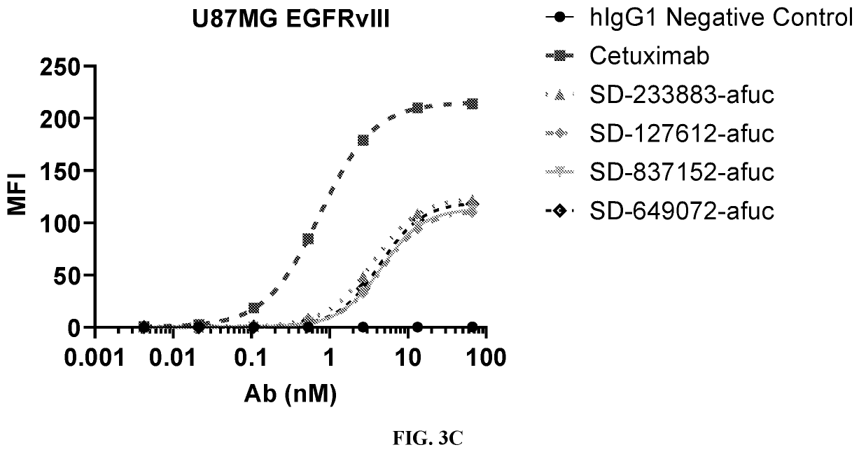

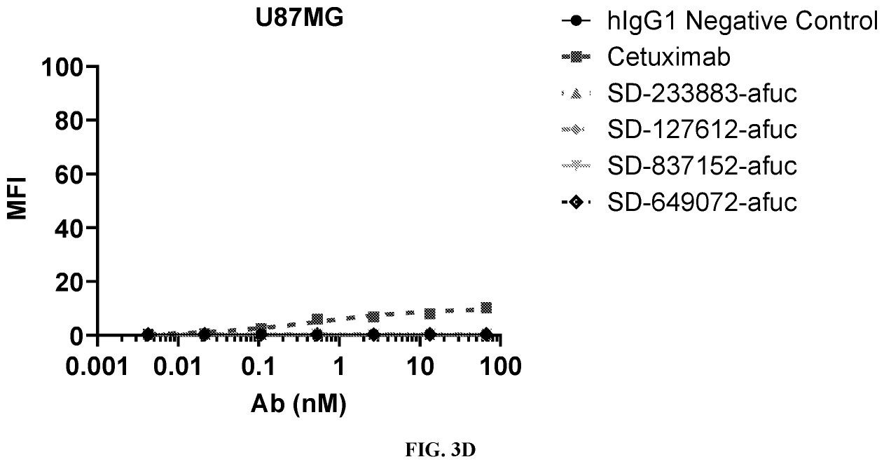

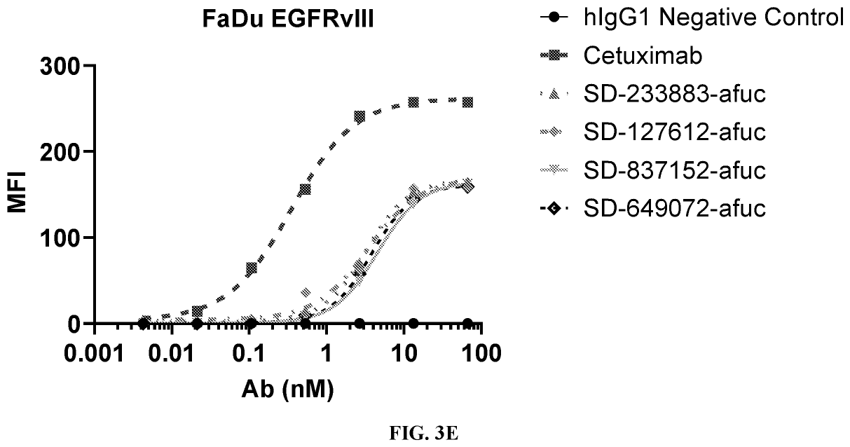

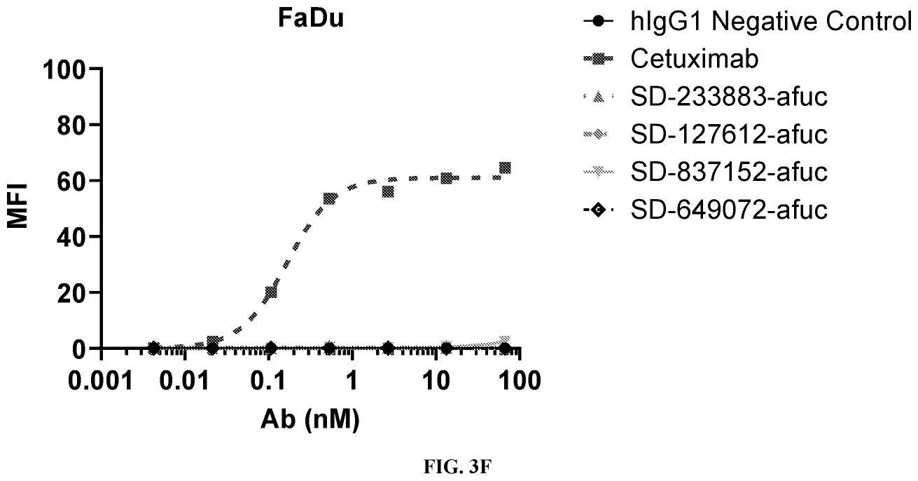

FIGS. 1A to 1C compare binding of the various antibodies by surface plasmon resonance binding to EGFRvIII and EGFR1, respectively (FIG. 1A, SD-127612-afuc, SD-233883-afuc, Cetuximab, SD-382591-afuc, hIgG1 negative control, SD-577776-afuc), (FIG. 3, SD-633416-afuc, SD-638526-afuc, SD-649072-afuc, SD-710726-afuc, SD-741396-afuc, SD-757052-afuc), and (FIG. 3C, SD-787077-afuc, SD-837152-afuc, SD-844257-afuc).

TABLE 2
Chimeric and humanized anti-EGFRvIII antibodies bind specifically to human
EGFRvIII but not to full length human EGFR1. Cetuximab recognizes both EGFR1
and EGFRvIII while a human IgG1 negative control antibody does not bind to
either. Kinetic parameters were measured by SPR using the Carterra LSA and
fitting the resulting sensorgrams to a 1:1 Langmuir binding model.

| | EGFRvIII | | | EGFR1 | | |
|---|---|---|---|---|---|---|
| | ka (s−1 | | | ka (s−1 | | |
| Clone | M−1) | kd (s−1) | KD (nM) | M−1) | kd (s−1) | KD (nM) |
| SD-127612-afuc | 4.20E+04 | 2.40E−03 | 57 | NB | NB | NB |
| SD-233883-afuc | 7.40E+04 | 3.00E−03 | 40 | NB | NB | NB |
| Cetuximab | 1.90E+06 | 1.10E−03 | 0.6 | 1.50E+06 | 1.50E−03 | 1 |
| SD-382591-afuc | 6.60E+04 | 8.70E−03 | 131 | NB | NB | NB |
| hIgG1 Negative | NB | NB | NB | NB | NB | NB |
| Control | | | | | | |
| SD-577776-afuc | 5.70E+04 | 5.80E−03 | 100 | NB | NB | NB |
| SD-633416-afuc | 6.70E+04 | 1.60E−02 | 234 | NB | NB | NB |
| SD-638526-afuc | 7.30E+04 | 8.80E−03 | 122 | NB | NB | NB |
| SD-649072-afuc | 5.10E+04 | 1.70E−03 | 33 | NB | NB | NB |
| SD-710726-afuc | 5.10E+04 | 4.30E−03 | 86 | NB | NB | NB |
| SD-741396-afuc | 6.60E+04 | 1.10E−02 | 159 | NB | NB | NB |
| SD-757052-afuc | 5.70E+04 | 6.00E−03 | 105 | NB | NB | NB |
| SD-787077-afuc | 7.10E+04 | 7.70E−03 | 108 | NB | NB | NB |
| SD-837152-afuc | 4.30E+04 | 2.60E−03 | 60 | NB | NB | NB |
| SD-844257-afuc | 5.30E+04 | 4.80E−03 | 90 | NB | NB | NB |

NB: no binding.

### Example 4. EGFRvIII and EGFR1 Binding ELISA

Binding ELISA. ELISA plates (Biolegend, Cat #423501) were first coated with 1 μg/mL of either human EGFRvIII (ACRO Biosciences, Cat #EGI-H52H4) or EGFR1 protein (ACRO Biosciences, Cat #EGR-H5222) in 50 mM carbonate buffer pH 9.5 (Teknova, Cat #S9225) overnight at 4° C. The next day, plates were washed three times with wash buffer [1×PBS with 0.1% Tween-20 (Teknova, Cat #P0207)] followed by the addition of blocking buffer [1×PBS, 1% BSA (Teknova, Cat #B0101)] for 1 hour at room temperature. After blocking, plates were washed three times with wash buffer followed by the addition of the anti-EGFRvIII or control antibodies in wash buffer at increasing concentrations (0.004-66.66 nM) for 1 hour at room temperature. After incubation, plates were washed three times with wash buffer followed by the addition of goat anti-human IgG-HRP secondary antibody (Biorad, Cat #STAR126P) at 1:2500 dilution in wash buffer for 1 hour at room temperature. After incubation with secondary antibody, plates were washed six times with wash buffer followed by the addition of TMB substrate (VWR, Cat #95059-154) for 5 minutes at room temperature. After TMB substrate incubation, ELISA stop solution (Thermo Fisher, Cat #SS04) was added to the plate at equal volume to the TMB substrate and the absorbance was read at 450 nm. The data was plotted using GraphPad Prism 9.3.0 software and the EC₅₀ values were calculated by the software.

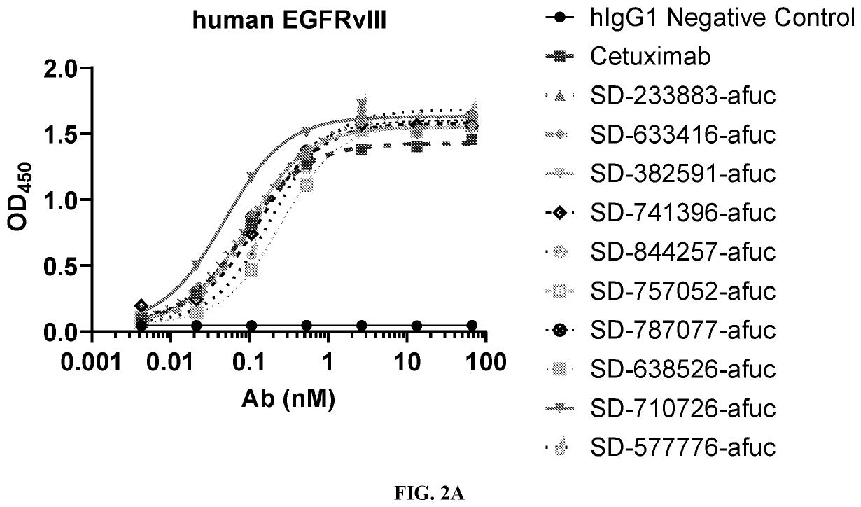

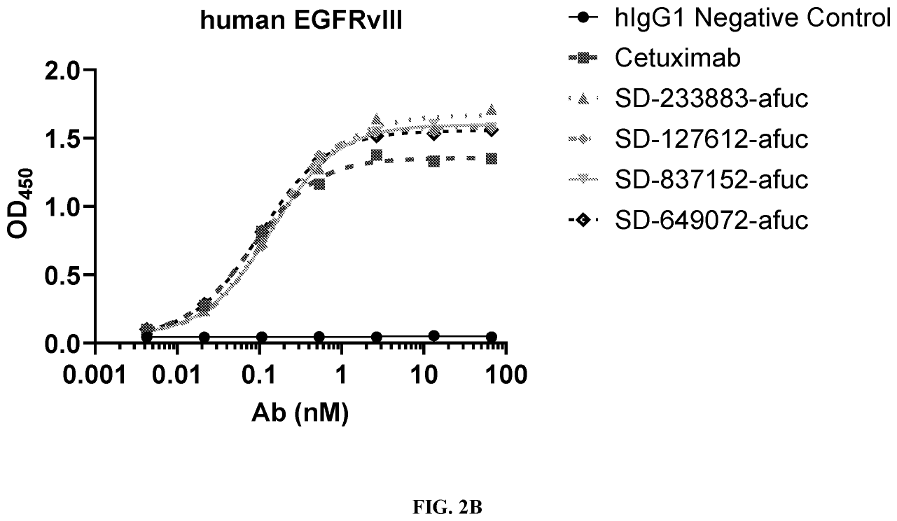

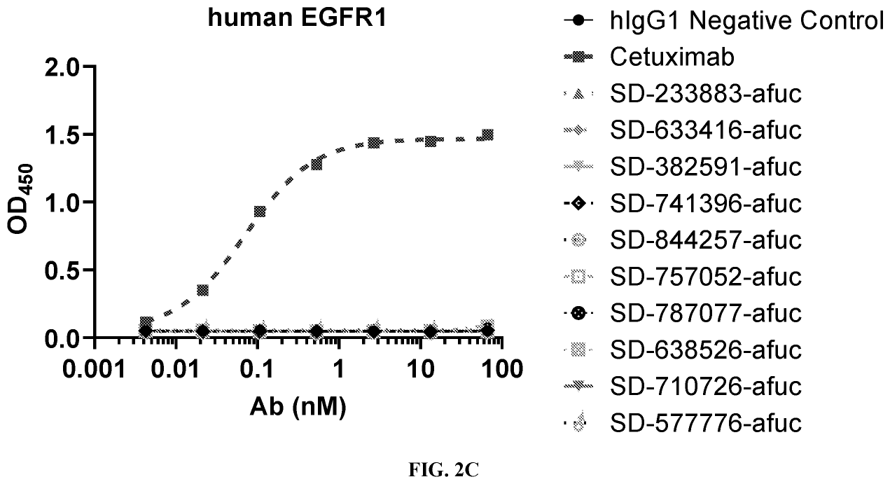

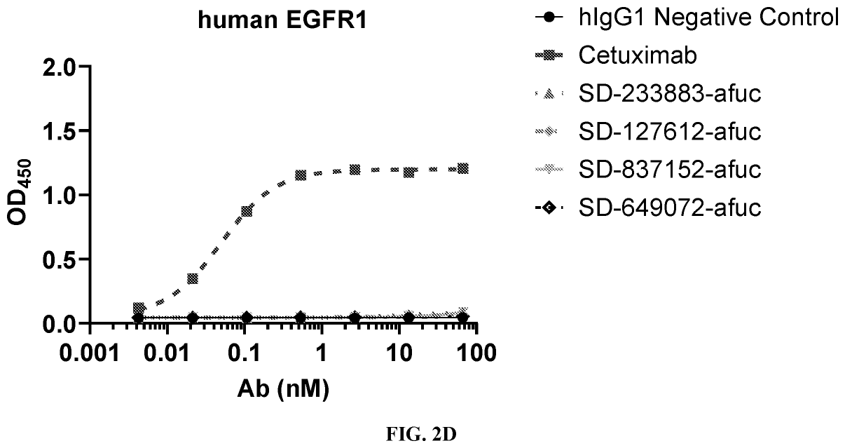

FIGS. 2A to 2D compare binding by ELISA to human EGFRvIII and EGFR1 by the hIgG1 isotype control antibody, Cetuximab, and the chimera and humanized antibodies of the present invention.

Chimera and humanized anti-EGFRvIII antibodies bind specifically to truncated human EGFRvIII but not the full length human EGFR1. ELISA assay showing afucosylated anti-EGFRvIII antibodies and cetuximab binding to immobilized human EGFRvIII in a concentration-dependent manner. Values plotted are measured absorbance at 450 nm wavelength. EC₅₀ values are averages of n=3 experiments. n/a, no activity.

TABLE 3
Chimera and humanized anti-EGFRvIII antibodies bind specifically to
truncated human EGFRvIII but not the full length human EGFR1.
EC50 (nM)

| Sample ID | EGFRvIII | EGFR1 |
|---|---|---|
| hIgG1 Negative Control | n/a | n/a |
| Cetuximab | 0.077 | 0.048 |
| SD-233883-afuc | 0.144 | n/a |
| SD-633416-afuc | 0.121 | n/a |
| SD-382591-afuc | 0.103 | n/a |
| SD-741396-afuc | 0.091 | n/a |
| SD-844257-afuc | 0.111 | n/a |
| SD-757052-afuc | 0.112 | n/a |
| SD-787077-afuc | 0.126 | n/a |
| SD-638526-afuc | 0.102 | n/a |
| SD-710726-afuc | 0.137 | n/a |
| SD-577776-afuc | 0.122 | n/a |
| SD-127612-afuc | 0.156 | n/a |
| SD-837152-afuc | 0.155 | n/a |
| SD-649072-afuc | 0.129 | n/a |

### Example 5. F98 EGFRvIII and EGFR1 Cell Binding Assay

F98npEGFRvIII (ATCC, Cat #CRL-2949) and F98 EGFR1 (ATCC, Cat #CRL-2948) cells were cultured in DMEM (Corning, Cat #10-013-CV) supplemented with 10% FBS (ATCC, Cat #30-2020) and 1× Penicillin-Streptomycin (Corning, Cat #30-002-CI), and 0.2 mg/ml G418 (Thermo Fisher, Cat #10131035). U87MG (ATCC, Cat #HTB-14) and U87MG-EGFRvIII (Genscript) cells were cultured in DMEM supplemented with 10% FBS, 1× Penicillin-Streptomycin, and 0.5 μg/mL Puromycin (for U87MG-EGFRvIII cells only, Gibco, Cat #A11138-03). FaDu (ATCC, Cat #HTB-43) and FaDu-EGFRvIII (Genscript) cells were cultured in EMEM (ATCC, Cat #30-2003) supplemented with 10% FBS, 1× Penicillin-Streptomycin, and 2 μg/mL Puromycin (for FaDu-EGFRvIII cells only).

For the cell binding assay, PBS (Corning, Cat #21-040-CV) supplemented with 2% FBS and 2 mM EDTA was used as the assay buffer. Target cells were counted then resuspended in their respective culture media, then seeded onto 96-well plates (VWR, Cat #89089-826) at 1×10⁵ cells/well and incubated on ice for 3 hours. After incubation, the plates were centrifuged and the supernatant was removed, then the cells were washed once with the assay buffer followed by centrifugation and wash removal. Following wash removal, the indicated antibodies were diluted in the assay buffer and added to the cells at increasing concentrations (0.004-66.66 nM) for 20 minutes on ice. Following incubation, the cells were washed with assay buffer followed by wash removal. Next, rat-anti-human IgG Fc Alexa Fluor 647 (BioLegend, Cat #410714) was added to the cells at 1:200 dilution in the assay buffer, for 20 minutes on ice. After incubation, the cells were washed once more with the assay buffer followed by wash removal. Next, DAPI (BioLegend, Cat #422801) was added to the cells at 1:5000 dilution in the assay buffer. Cell binding was analyzed on a Miltenyi MACSQuant 16 flow cytometer. Flow cytometry data were analyzed with the FlowJo flow cytometry analysis software. For making graphs and calculation of the EC₅₀ values GraphPad Prism 9.3.0 was used.

FIGS. 3A to 3F compare binding by chimera and humanized anti-EGFRvIII antibodies of the present invention. The antibodies listed bind specifically to human EGFRvIII but not to wild-type human EGFR1. FACS analysis shows anti-EGFRvIII antibodies binding specifically to F98 rat glioblastoma (FIG. 3A), U87MG human glioblastoma (FIG. 3C), and FaDu human head and neck cancer cells (FIG. 3E) that are overexpressing human EGFRvIII. No binding was detected to F98 cells overexpressing wild-type human EGFR1 cells (FIG. 3B) or to wild-type U87MG (FIG. 3D) and FaDu (FIG. 3F) cells. Values plotted are Median Fluorescent Intensity. EC₅₀ values are averages of n=3 experiments. n/a, no activity.

TABLE 4
Chimera and humanized anti-EGFRvIII antibodies bind
specifically to human EGFRvIII but not to the human EGFR1
overexpressed in F98 rat glioblastoma cell line.
EC50 (nM)

| Sample ID | F98 EGFRvIII | F98 EGFR1 |
|---|---|---|
| hIgG1 Negative Control | n/a | n/a |
| Cetuximab | 0.223 | 0.217 |
| SD-233883-afuc | 2.066 | n/a |
| SD-633416-afuc | 13.760 | n/a |
| SD-382591-afuc | 4.134 | n/a |
| SD-741396-afuc | 5.122 | n/a |
| SD-844257-afuc | 3.854 | n/a |
| SD-757052-afuc | 4.171 | n/a |
| SD-787077-afuc | 3.908 | n/a |
| SD-638526-afuc | 3.210 | n/a |
| SD-710726-afuc | 4.020 | n/a |
| SD-577776-afuc | 3.457 | n/a |

TABLE 5
Chimera and humanized anti-EGFRvIII antibodies bind
specifically to human EGFRvIII overexpressed in FaDu
human head and neck cancer cells but not to wild type FaDu cells.
EC50 (nM)

| Sample ID | FaDu-EGFRvIII | FaDu |
|---|---|---|
| hIgG1 Negative Control | n/a | n/a |
| Cetuximab | 0.401 | 0.197 |
| SD-233883-afuc | 3.127 | n/a |
| SD-127612-afuc | 5.269 | n/a |
| SD-837152-afuc | 4.976 | n/a |
| SD-649072-afuc | 3.857 | n/a |

TABLE 6
Chimera and humanized anti-EGFRvIII antibodies bind
specifically to human EGFRvIII overexpressed in U87MG
human glioblastoma cells but not to wild type U87MG cells.
EC50 (nM)

| Sample ID | U87MG-EGFRvIII | U87MG |
|---|---|---|
| hIgG1 Negative Control | n/a | n/a |
| Cetuximab | 0.647 | 0.301 |
| SD-233883-afuc | 4.048 | n/a |
| SD-127612-afuc | 6.055 | n/a |
| SD-837152-afuc | 5.750 | n/a |
| SD-649072-afuc | 4.331 | n/a |

### Example 7. In Vitro ADCC Assay

F98npEGFRvIII (ATCC, Cat #CRL-2949) and F98 EGFR1 (ATCC, Cat #CRL-2948) cells were cultured in DMEM (Corning, Cat #10-013-CV) supplemented with 10% FBS (ATCC, Cat #30-2020) and 1× Penicillin-Streptomycin (Corning, Cat #30-002-CI), and 0.2 mg/ml G418 (Thermo Fisher, Cat #10131035). U87MG (ATCC, Cat #HTB-14) and U87MG-EGFRvIII (Genscript) cells were cultured in DMEM supplemented with 10% FBS, 1× Penicillin-Streptomycin, and 0.5 μg/mL Puromycin (for U87MG-EGFRvIII cells only, Gibco, Cat #A11138-03). FaDu (ATCC, Cat #HTB-43) and FaDu-EGFRvIII (Genscript) cells were cultured in EMEM (ATCC, Cat #30-2003) supplemented with 10% FBS, 1× Penicillin-Streptomycin, and 2 μg/mL Puromycin (for FaDu-EGFRvIII cells only)

For ADCC evaluation, PBMC frozen stock purchased from STEMCELL (Cat #70025) was thawed in RPMI media supplied with 10% FBS, 1× Penicillin-Streptomycin (Corning, Cat #30-002-CI), 5 ng/ml IL2 (Miltenyi Biotec, Cat #130-097-743) and incubated in tissue culture incubator overnight.

Target cells were counted, and cell viability was assessed. Cells were first stained with CFSE dye (Thermo Fisher, Cat #C34554) for 10 minutes at room temperature then washed once with growth media. Washed cells were resuspended in growth media to the density of 1×10⁶ cells/mL, then 1×10⁴ cells were seeded per well onto 96 well plates (Fisher Scientific, Cat #07-200-89) and incubated in tissue culture incubator overnight. After incubating the cells, the growth media in assay plate was replaced with assay media consisting of RPMI 1640 media supplemented with 10% FBS, 1% Penicillin-Streptomycin, 5 ng/mL IL2 (Miltenyi Biotec, Cat #130-097-743). Control or anti-EGFRvIII antibodies in assay media were added to the cells at increasing concentrations (0.0004 to 6.66 nM for FaDu and U87 cells, 0.0002 to 3.33 nM for F98 cells) for 10 minutes at 37° C., 5% CO₂. After that, 2×10⁵ peripheral blood mononuclear cells (PBMC) were added per well of 96 well plate. Cells and antibodies were incubated at 37° C. in 5% CO₂ incubator for 24 hrs. Samples were stained with LIVE/DEAD™ Fixable Aqua Dead Cell Stain Kit (Thermo Fisher, Cat #L34957) and analyzed using MACSQuant 16 flow cytometer. Live cells were gated (aqua−, CFSE+) and dead cells were gated (aqua+, CFSE+). The ratio of dead cells to the total target cells was used to determine the percentage of cell lysis. Flow cytometry data were analyzed with the FlowJo flow cytometry analysis software. For making graphs and calculation of the EC₅₀ values GraphPad Prism 9.3.0 was used.

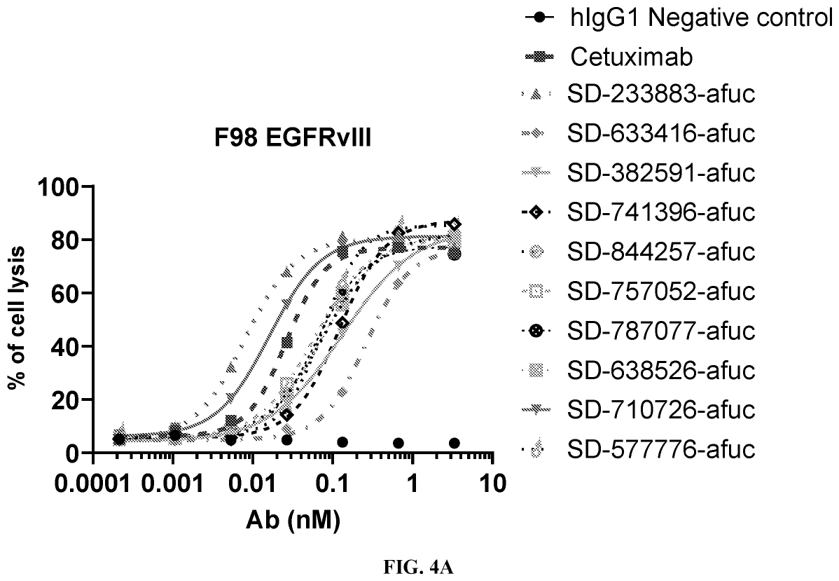

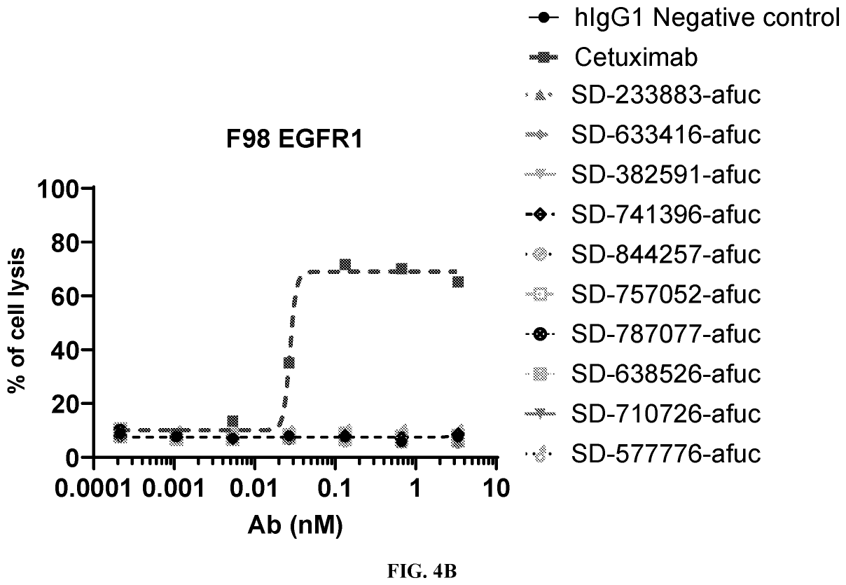

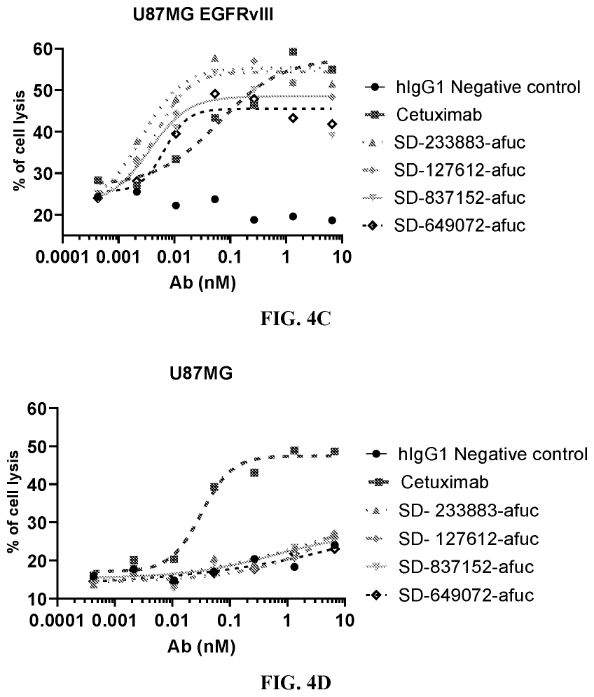

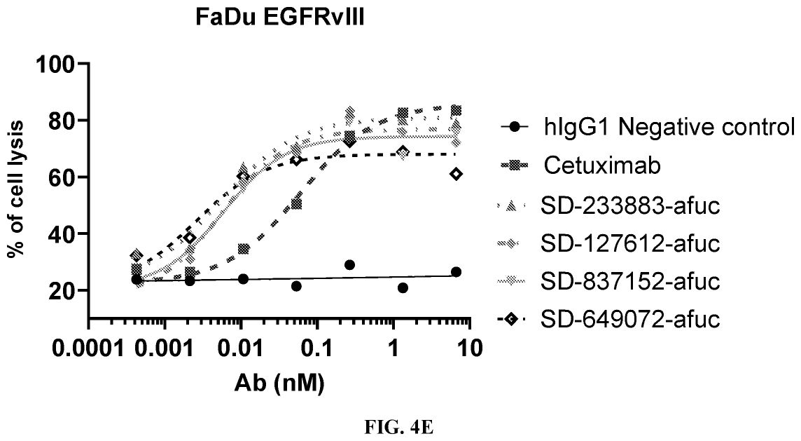

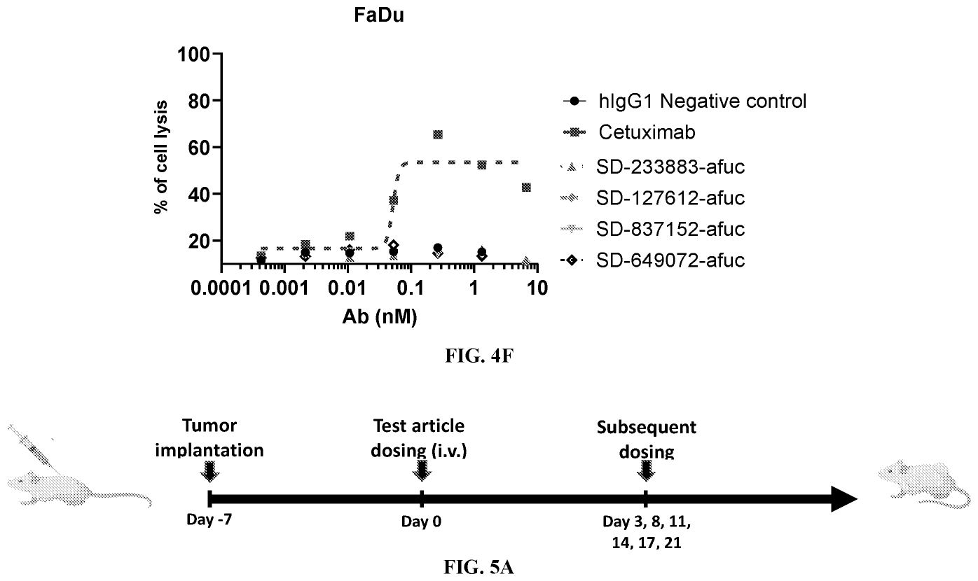

FIG. 4A to 4F show chimera and humanized anti-EGFRvIII antibodies of the present invention demonstrating potent ADCC activity against F98 (FIG. 4A), U87MG (FIG. 4C), FaDu cells (FIG. 4E) overexpressing human EGFRvIII but not against human EGFR1 expressing F98 (FIG. 4B), wild type U87MG (FIG. 4D) and FaDu (FIG. 4F) cells. The ratio of dead cells to the total cells was used to determine the percentage of cell lysis. EC₅₀ values are averages of n=1-5 experiments. n/a, no activity.

TABLE 7
Chimera and humanized anti-EGFRvIII antibodies demonstrated potent
ADCC activity against F98.
EC50 (nM)

| Sample ID | F98 EGFRvIII | F98 EGFR1 |
|---|---|---|
| hIgG1 Negative Control | n/a | n/a |
| Cetuximab | 0.0158 | 0.01858 |
| SD-233883-afuc | 0.0082 | n/a |
| SD-633416-afuc | 0.2895 | n/a |
| SD-382591-afuc | 0.1058 | n/a |
| SD-741396-afuc | 0.1233 | n/a |
| SD-844257-afuc | 0.0738 | n/a |
| SD-757052-afuc | 0.0562 | n/a |
| SD-787077-afuc | 0.0605 | n/a |
| SD-638526-afuc | 0.1113 | n/a |
| SD-710726-afuc | 0.0168 | n/a |
| SD-577776-afuc | 0.0687 | n/a |

TABLE 8
Chimera and humanized anti-EGFRvIII antibodies demonstrated potent
ADCC activity against human EGFRvIII overexpressed in U87MG human
glioblastoma cells but not to wild type U87MG cells.
EC50 (nM)

| Sample ID | U87MG-EGFRvIII | U87MG |
|---|---|---|
| hIgG1 Negative Control | n/a | n/a |
| Cetuximab | 0.0632 | 0.0318 |
| SD-233883-afuc | 0.0026 | n/a |
| SD-127612-afuc | 0.0047 | n/a |
| SD-837152-afuc | 0.0036 | n/a |
| SD-649072-afuc | 0.0066 | n/a |

TABLE 9
Chimera and humanized anti-EGFRvIII antibodies demonstrated
potent ADCC activity against human EGFRvIII overexpressed in
FaDu human head and neck cancer cells but not to wild type FaDu cells.
EC50 (nM)

| Sample ID | FaDu-EGFRvIII | FaDu |
|---|---|---|
| hIgG1 Negative Control | n/a | n/a |
| Cetuximab | 0.0551 | 0.0443 |
| SD-233883-afuc | 0.0039 | n/a |
| SD-127612-afuc | 0.0051 | n/a |
| SD-837152-afuc | 0.0058 | n/a |
| SD-649072-afuc | 0.0033 | n/a |

In vivo Efficacy Assay. Nine-week-old female nude mice (Charles River Laboratories, Cat #088Nu/Nu) were used in this assay. 2×10⁶ FaDu-EGFRvIII cells in 100 μl the mixture of PBS and MatriGel (Corning, Cat #354234) (v:v=1:1) were inoculated to the left upper side of each nude mouse by subcutaneous injection. Tumor growth and mouse body weight were monitored twice a week. For each individual tumor, the longest longitudinal diameter as length and the widest transverse diameter as width were measured by using a Traceable Digital Caliper (VWR, Cat #62379-531). Tumor volume (TV) was then calculated by the formula TV=[length×(width)2]/2. Mice were grouped randomly when the average tumor volume reached 152-153 mm³, and each group contained 10 mice. Each of hIgG1 negative control, Cetuximab and SD-233883-afuc was made in a stock solution of 3 mg/ml in PBS. For each mouse, the volume of administered antibody was calculated by the formula Volume (μl)=Mouse body weight (g)×10 μl/g. Antibody drug were administered twice a week for 7 times total (biw×7) via the intravenous route. After initial drug treatment, the percentage change of each tumor was calculated by the formula: TV change %=[(TV−TVday0)/TVday0]×100.

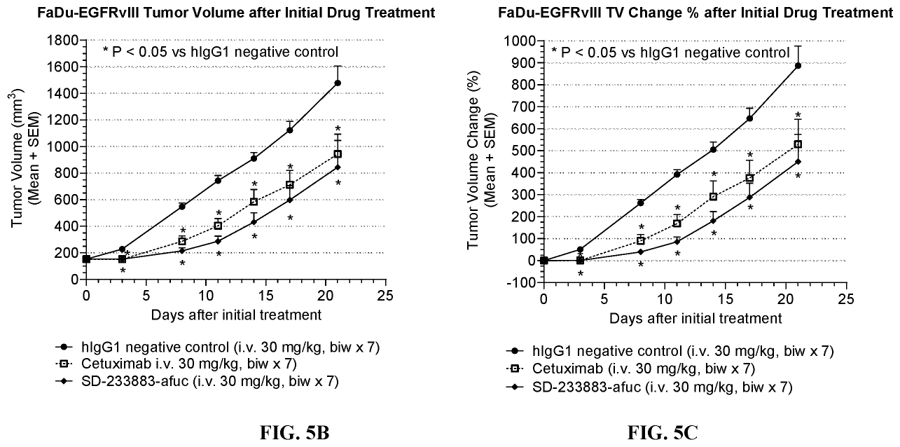

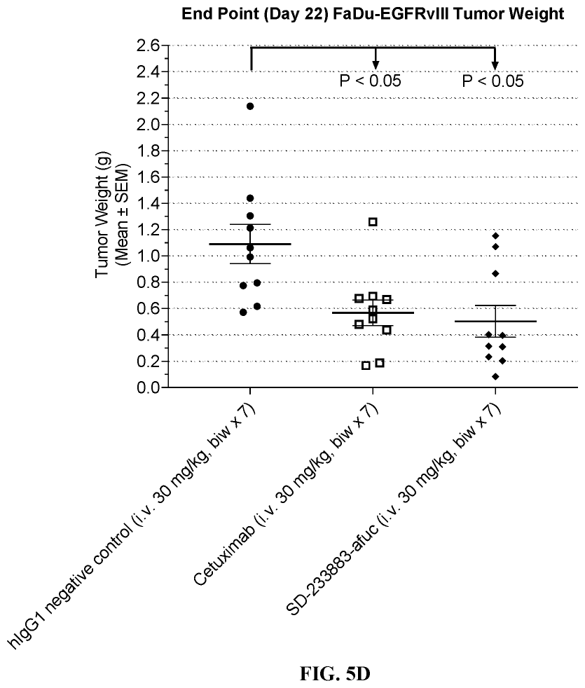

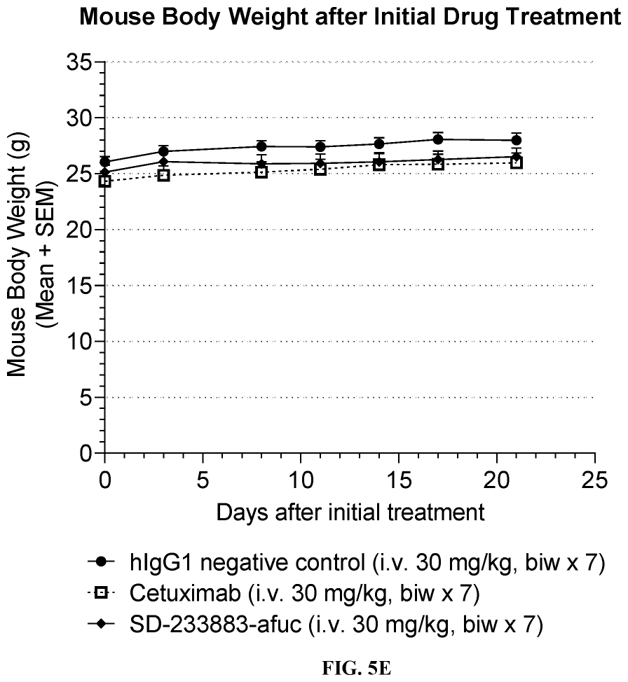

FIGS. 5A to 5E show: FIG. 5A study design for in vivo efficacy of anti-EGFRvIII antibody SD-233883-afuc against FaDu EGFRvIII tumor cells in a nude mice model. FIG. 5B (FaDu-EGFRvIII tumor volume after initial drug treatment) and FIG. 5C (FaDu-EGFRvIII tumor volume change % after initial drug treatment) show that SD-233883-afuc significantly inhibited FaDu-EGFRvIII tumor growth throughout the entire observation time window (T-test, P<0.05) in comparison with hIgG1 negative control. FIG. 5D shows at the endpoint (day 22) of the study, FaDu-EGFRvIII tumor weight was significantly reduced after the treatment of Cetuximab or SD-233883-afuc (T-test, P<0.05) in comparison with hIgG1 negative control. FIG. 5E shows mouse body weight after initial drug treatment.

Provided herein are antibodies that bind to the truncated EGFRvIII. These antibodies are referred to herein as anti-EGFRvIII antibodies. A number of discovery strategies have been employed to obtain the exemplary antibodies of the disclosure, further discussed below.

The skilled artisan will recognize that antibodies which exhibit little or no binding to a target antigen can be described as having a low affinity, and a high equilibrium dissociation constant (K_{D}) for the target antigen. The skilled artisan will also recognize that antibodies which exhibit little or no binding to a collective assembly of target antigenic epitopes can be described as having a low avidity, and a high equilibrium dissociation constant (K_{D}) for the collective assembly of target antigenic epitopes.

In some embodiments, provided herein are anti-EGFRvIII antibodies having a binding affinity (K_{D}) to EGFRvIII of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 pM to about 5 pM.

In some embodiments, anti-EGFRvIII antibodies have a binding avidity (EC₅₀) to EGFRvIII of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, anti-EGFRvIII antibodies have a half maximal effective concentration (EC₅₀) to EGFRvIII of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

In some embodiments, the anti-EGFRvIII antibody is a full length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of the humanized antibody is from a human IgG1, human IgG2, human IgG3, or human IgG4.

### Example 8. Exemplary Anti-EGFRvIII Antibodies—CDR Sequences

Provided herein are sequences for exemplary anti-EGFRvIII antibodies of the disclosure. Included are complementarity determining region (CDR) sequences and the variable heavy and light domain sequences (VH, VL) that constitute the EGFRvIII antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 10 provides exemplary CDR combinations of antibodies of the disclosure.

TABLE 10
Anti-EGFRvIII name and type.

| Sample ID | Description |
|---|---|
| SD-233883-afuc | Chimera |
| SD-633416-afuc | Humanized |
| SD-382591-afuc | Humanized |
| SD-741396-afuc | Humanized |
| SD-844257-afuc | Humanized |
| SD-757052-afuc | Humanized |
| SD-787077-afuc | Humanized |
| SD-638526-afuc | Humanized |
| SD-710726-afuc | Humanized |
| SD-577776-afuc | Humanized |
| SD-127612-afuc | Humanized |
| SD-837152-afuc | Humanized |
| SD-649072-afuc | Humanized, affinity matured |

TABLE 11
Full-length Humanized and Chimeric anti-EGFRvIII antibodies.

| | SEQ | |
|---|---|---|
| Name | ID NO | Sequence |
| SD- | 1 | EVQLQQFGAELVKPGASVKLSCKASGYTFTSYDINWVRQ |
| 233883_VH | | RPEQGLEWIGWIFPGDGTSKYNEKFKGKATLSTDKSSSTA |
| | | YMQLSRLTIEDSAVYFCARRLGSHWGQGSTLTVSS |
| SD- | 2 | DIVLTQSPASLAVSLGQRATISCKASQSVDSDGDSYMNWY |
| 233883_VL | | QQKPGQPPKLLIYGASNLESGIPARFSGSGSGTDFSLNIHPV |
| | | EEEDAATYYCQQSHEYPFTFGGGTKLEIK |
| SD- | 3 | GYTFTSYD |
| 233883_hCDR1 | | |
| SD- | 4 | IFPGDGTS |
| 233883_hCDR2 | | |
| SD- | 5 | ARRLGSH |
| 233883_hCDR3 | | |
| SD- | 6 | QSVDSDGDSY |
| 233883_1CDR1 | | |
| SD- | 7 | GAS |
| 233883_1CDR2 | | |
| SD- | 8 | QQSHEYPFT |
| 233883_1CDR3 | | |
| SD- | 9 | GAAGTGCAACTCCAGCAATTCGGTGCCGAGCTTGTGAA |
| 233883_VH_ | | GCCTGGGGCCTCAGTGAAGCTGAGCTGCAAGGCCTCCG |
| DNA | | GATATACCTTCACCTCCTACGATATCAACTGGGTCCGGC |
| | | AGAGGCCGGAACAGGGCCTGGAGTGGATCGGTTGGATC |
| | | TTCCCCGGCGACGGGACCTCGAAGTACAACGAAAAGTT |
| | | CAAGGGAAAAGCAACGCTGTCCACCGACAAGTCCTCAT |
| | | CCACTGCGTACATGCAGCTGTCCCGCCTGACTATTGAGG |
| | | ACTCCGCTGTGTACTTTTGTGCCCGGAGACTCGGAAGCC |
| | | ACTGGGGACAGGGCAGCACCTTGACTGTGTCGTCG |
| SD- | 10 | GATATTGTGCTGACTCAAAGCCCTGCGTCCCTGGCTGTC |
| 233883_VL_ | | TCCCTGGGACAGCGCGCCACCATTTCATGCAAAGCCTC |
| DNA | | CCAGTCCGTGGACAGCGACGGGGACAGCTATATGAACT |
| | | GGTACCAGCAGAAGCCCGGGCAGCCTCCGAAGCTGCTT |
| | | ATCTACGGTGCCTCCAACTTGGAGTCGGGAATCCCCGC |
| | | ACGGTTCTCCGGATCGGGCTCCGGAACTGACTTCTCGCT |
| | | CAACATCCACCCAGTGGAAGAAGAAGATGCCGCCACCT |
| | | ACTACTGTCAGCAATCACATGAGTACCCGTTTACCTTCG |
| | | GTGGCGGCACCAAGCTCGAGATCAAG |
| SD- | 11 | EVQLVQSGAEVKKPGASVKVSCKASGYTFTSYDINWVRQ |
| 633416_VH | | APGQGLEWMGWIFPGDGTSKYAQKFQGRVTMTRDTSTST |
| | | VYMELSSLRSEDTAVYYCARRLGSHWGQGTTVTVSS |
| SD- | 12 | DIQMTQSPSSLSASVGDRVTITCKASQSVDSDGDSYMNWY |
| 633416_VL | | QQKPGKAPKLLIYGASNLESGVPSRFSGSGSGTDFTLTISSL |
| | | QPEDFATYYCQQSHEYPFTFGQGTKLEIK |
| SD- | 13 | GYTFTSYD |
| 633416_hCDR1 | | |
| SD- | 14 | IFPGDGTS |
| 633416_hCDR2 | | |
| SD- | 15 | ARRLGSH |
| 633416_hCDR3 | | |
| SD- | 16 | QSVDSDGDSY |
| 633416_1CDR1 | | |
| SD- | 17 | GAS |
| 633416_1CDR2 | | |
| SD- | 18 | QQSHEYPFT |
| 633416_1CDR3 | | |
| SD- | 19 | GAAGTGCAACTGGTGCAAAGCGGTGCCGAAGTCAAGA |
| 633416_VH_ | | AGCCCGGAGCCTCAGTGAAAGTGTCCTGCAAGGCTTCG |
| DNA | | GGCTACACCTTCACCTCCTACGACATTAACTGGGTCAGA |
| | | CAGGCACCTGGACAGGGCCTGGAGTGGATGGGCTGGAT |
| | | CTTCCCGGGCGACGGAACTTCGAAATACGCCCAGAAGT |
| | | TTCAGGGTCGCGTGACTATGACTCGGGATACTTCCACCT |
| | | CCACCGTGTACATGGAACTCAGCTCCCTTCGGTCCGAG |
| | | GACACCGCCGTCTACTATTGTGCGAGGAGACTGGGGTC |
| | | ACACTGGGGACAGGGGACGACCGTGACCGTGTCGAGC |
| SD- | 20 | GATATTCAGATGACGCAGAGCCCCTCGTCCCTCTCCGCT |
| 633416_VL_ | | TCCGTGGGAGATCGCGTCACCATTACTTGCAAAGCCAG |
| DNA | | CCAGTCCGTGGACTCGGACGGAGACTCCTACATGAACT |
| | | GGTACCAGCAGAAGCCAGGAAAGGCCCCGAAGCTGCTT |
| | | ATCTACGGGGCCTCCAACTTGGAATCGGGAGTGCCTTC |
| | | ACGGTTCTCTGGTTCCGGCTCCGGCACTGACTTTACCCT |
| | | GACCATCAGCAGCCTGCAGCCGGAGGACTTCGCGACTT |
| | | ACTACTGCCAACAGTCACACGAATATCCCTTCACCTTCG |
| | | GCCAAGGGACCAAGCTGGAGATCAAG |
| SD- | 21 | EVQLVQSGAEVKKPGASVKVSCKASGYTFTSYDINWVRQ |
| 382591_VH | | APGQGLEWMGWIFPGDGTSKYAQKFQGRVTMTTDTSTST |
| | | AYMELSSLRSEDTAVYYCARRLGSHWGQGTTVTVSS |
| SD- | 22 | DIQLTQSPSSLSASVGDRVTITCKASQSVDSDGDSYMNWY |
| 382591_VL | | QQKPGKAPKLLIYGASNLESGVPSRFSGSGSGTDFTLTISSL |
| | | QPEDFATYYCQQSHEYPFTFGQGTKLEIK |
| SD- | 23 | GYTFTSYD |
| 382591_hCDR1 | | |
| SD- | 24 | IFPGDGTS |
| 382591_hCDR2 | | |
| SD- | 25 | ARRLGSH |
| 382591_hCDR3 | | |
| SD- | 26 | QSVDSDGDSY |
| 382591_1CDR1 | | |
| SD- | 27 | GAS |
| 382591_1CDR2 | | |
| SD- | 28 | QQSHEYPFT |
| 382591_1CDR3 | | |
| SD- | 29 | GAAGTGCAGCTGGTGCAGTCAGGCGCCGAGGTCAAGAA |
| 382591_VH_ | | GCCCGGAGCAAGCGTGAAAGTGTCCTGCAAGGCCTCAG |
| DNA | | GGTACACTTTCACCTCCTATGACATCAACTGGGTCAGAC |
| | | AGGCTCCGGGACAAGGGCTCGAATGGATGGGTTGGATT |
| | | TTCCCTGGCGACGGCACATCGAAATACGCGCAGAAGTT |
| | | TCAGGGACGCGTGACCATGACCACCGACACGTCCACTT |
| | | CCACTGCCTACATGGAACTGAGCTCGCTGCGGTCCGAG |
| | | GATACCGCCGTGTACTACTGTGCCCGGAGGCTTGGCAG |
| | | CCACTGGGGTCAAGGAACCACCGTGACTGTGTCCTCG |
| SD- | 30 | GATATTCAGCTGACGCAGAGCCCCTCGTCCCTCTCCGCT |
| 382591_VL_ | | TCCGTGGGAGATCGCGTCACCATTACTTGCAAAGCCAG |
| DNA | | CCAGTCCGTGGACTCGGACGGAGACTCCTACATGAACT |
| | | GGTACCAGCAGAAGCCAGGAAAGGCCCCGAAGCTGCTT |
| | | ATCTACGGGGCCTCCAACTTGGAATCGGGAGTGCCTTC |
| | | ACGGTTCTCTGGTTCCGGCTCCGGCACTGACTTTACCCT |
| | | GACCATCAGCAGCCTGCAGCCGGAGGACTTCGCGACTT |
| | | ACTACTGCCAACAGTCACACGAATATCCCTTCACCTTCG |
| | | GCCAAGGGACCAAGCTGGAGATCAAG |
| SD- | 31 | EVQLVQSGAEVKKPGASVKVSCKASGYTFTSYDINWVRQ |
| 741396_VH | | APGQGLEWMGWIFPGDGTSKYAQKFQGRVTMTTDTSTST |
| | | AYMELSSLRSEDTAVYYCARRLGSHWGQGTTVTVSS |
| SD- | 32 | DIQLTQSPSSLSASVGDRATITCKASQSVDSDGDSYMNWY |
| 741396_VL | | QQKPGKAPKLLIYGASNLESGIPSRFSGSGSGTDFTLTISSV |
| | | QPEDFATYYCQQSHEYPFTFGQGTKLEIK |
| SD- | 33 | GYTFTSYD |
| 741396_hCDR1 | | |
| SD- | 34 | IFPGDGTS |
| 741396_hCDR2 | | |
| SD- | 35 | ARRLGSH |
| 741396_hCDR3 | | |
| SD- | 36 | QSVDSDGDSY |
| 741396_1CDR1 | | |
| SD- | 37 | GAS |
| 741396_1CDR2 | | |
| SD- | 38 | QQSHEYPFT |
| 741396_1CDR3 | | |
| SD- | 39 | GAAGTGCAGCTGGTGCAGTCAGGCGCCGAGGTCAAGAA |
| 741396_VH_ | | GCCCGGAGCAAGCGTGAAAGTGTCCTGCAAGGCCTCAG |
| DNA | | GGTACACTTTCACCTCCTATGACATCAACTGGGTCAGAC |
| | | AGGCTCCGGGACAAGGGCTCGAATGGATGGGTTGGATT |
| | | TTCCCTGGCGACGGCACATCGAAATACGCGCAGAAGTT |
| | | TCAGGGACGCGTGACCATGACCACCGACACGTCCACTT |
| | | CCACTGCCTACATGGAACTGAGCTCGCTGCGGTCCGAG |
| | | GATACCGCCGTGTACTACTGTGCCCGGAGGCTTGGCAG |
| | | CCACTGGGGTCAAGGAACCACCGTGACTGTGTCCTCG |
| SD- | 40 | GATATTCAGTTGACCCAGTCCCCGAGCTCACTGTCCGCT |
| 741396_VL_ | | TCCGTGGGTGATCGCGCCACTATCACGTGTAAAGCGTC |
| DNA | | CCAGAGCGTCGACTCCGACGGGGACTCCTACATGAACT |
| | | GGTATCAGCAGAAGCCCGGAAAGGCCCCTAAGCTCCTG |
| | | ATCTACGGCGCATCCAACCTGGAAAGCGGAATCCCCTC |
| | | GCGGTTCTCGGGAAGCGGCTCTGGGACCGACTTCACCC |
| | | TTACTATCTCATCGGTGCAACCGGAGGACTTCGCCACCT |
| | | ACTACTGCCAACAGTCCCACGAATACCCATTCACCTTTG |
| | | GACAAGGCACTAAGCTGGAGATTAAG |
| SD- | 41 | EVQLVQSGAEVKKPGASVKLSCKASGYTFTSYDINWVRQ |
| 844257_VH | | APGQGLEWIGWIFPGDGTSKYAQKFQGRATLTTDTSTSTA |
| | | YMELSSLRSEDTAVYYCARRLGSHWGQGTTLTVSS |
| SD- | 42 | DIQLTQSPSSLSASVGDRVTITCKASQSVDSDGDSYMNWY |
| 844257_VL | | QQKPGKAPKLLIYGASNLESGVPSRFSGSGSGTDFTLTISSL |
| | | QPEDFATYYCQQSHEYPFTFGQGTKLEIK |
| SD- | 43 | GYTFTSYD |
| 844257_hCDR1 | | |
| SD- | 44 | IFPGDGTS |
| 844257_hCDR2 | | |
| SD- | 45 | ARRLGSH |
| 844257_hCDR3 | | |
| SD- | 46 | QSVDSDGDSY |
| 844257_1CDR1 | | |
| SD- | 47 | GAS |
| 844257_1CDR2 | | |
| SD- | 48 | QQSHEYPFT |
| 844257_1CDR3 | | |
| SD- | 49 | GAAGTGCAGCTCGTGCAGTCCGGAGCCGAAGTCAAGAA |
| 844257_VH_ | | GCCGGGAGCCTCAGTGAAGCTCAGCTGCAAGGCCTCGG |
| DNA | | GCTACACCTTCACTTCCTACGACATTAACTGGGTCAGAC |
| | | AGGCACCTGGACAAGGCTTGGAGTGGATCGGATGGATC |
| | | TTTCCCGGCGATGGGACTAGCAAATACGCCCAGAAGTT |
| | | CCAGGGTCGCGCGACTCTGACCACCGACACCTCCACCT |
| | | CAACCGCGTATATGGAACTGTCCTCCCTTCGGTCGGAG |
| | | GACACTGCCGTGTACTACTGCGCTAGAAGGCTGGGCAG |
| | | CCACTGGGGTCAAGGGACCACACTGACGGTGTCGTCC |
| SD- | 50 | GATATTCAGCTGACGCAGAGCCCCTCGTCCCTCTCCGCT |
| 844257_VL_ | | TCCGTGGGAGATCGCGTCACCATTACTTGCAAAGCCAG |
| DNA | | CCAGTCCGTGGACTCGGACGGAGACTCCTACATGAACT |
| | | GGTACCAGCAGAAGCCAGGAAAGGCCCCGAAGCTGCTT |
| | | ATCTACGGGGCCTCCAACTTGGAATCGGGAGTGCCTTC |
| | | ACGGTTCTCTGGTTCCGGCTCCGGCACTGACTTTACCCT |
| | | GACCATCAGCAGCCTGCAGCCGGAGGACTTCGCGACTT |
| | | ACTACTGCCAACAGTCACACGAATATCCCTTCACCTTCG |
| | | GCCAAGGGACCAAGCTGGAGATCAAG |
| SD- | 51 | EVQLVQSGAEVKKPGASVKLSCKASGYTFTSYDINWVRQ |
| 757052_VH | | APGQGLEWIGWIFPGDGTSKYAQKFQGRATLTTDTSTSTA |
| | | YMELSSLRSEDTAVYYCARRLGSHWGQGTTLTVSS |
| SD- | 52 | DIQLTQSPSSLSASVGDRATITCKASQSVDSDGDSYMNWY |
| 757052_VL | | QQKPGKAPKLLIYGASNLESGIPSRFSGSGSGTDFTLTISSV |
| | | QPEDFATYYCQQSHEYPFTFGQGTKLEIK |
| SD- | 53 | GYTFTSYD |
| 757052_hCDR1 | | |
| SD- | 54 | IFPGDGTS |
| 757052_hCDR2 | | |
| SD- | 55 | ARRLGSH |
| 757052_hCDR3 | | |
| SD- | 56 | QSVDSDGDSY |
| 757052_1CDR1 | | |
| SD- | 57 | GAS |
| 757052_1CDR2 | | |
| SD- | 58 | QQSHEYPFT |
| 757052_1CDR3 | | |
| SD- | 59 | GAAGTGCAGCTCGTGCAGTCCGGAGCCGAAGTCAAGAA |
| 757052_VH_ | | GCCGGGAGCCTCAGTGAAGCTCAGCTGCAAGGCCTCGG |
| DNA | | GCTACACCTTCACTTCCTACGACATTAACTGGGTCAGAC |
| | | AGGCACCTGGACAAGGCTTGGAGTGGATCGGATGGATC |
| | | TTTCCCGGCGATGGGACTAGCAAATACGCCCAGAAGTT |
| | | CCAGGGTCGCGCGACTCTGACCACCGACACCTCCACCT |
| | | CAACCGCGTATATGGAACTGTCCTCCCTTCGGTCGGAG |
| | | GACACTGCCGTGTACTACTGCGCTAGAAGGCTGGGCAG |
| | | CCACTGGGGTCAAGGGACCACACTGACGGTGTCGTCC |
| SD- | 60 | GATATTCAGTTGACCCAGTCCCCGAGCTCACTGTCCGCT |
| 757052_VL_ | | TCCGTGGGTGATCGCGCCACTATCACGTGTAAAGCGTC |
| DNA | | CCAGAGCGTCGACTCCGACGGGGACTCCTACATGAACT |
| | | GGTATCAGCAGAAGCCCGGAAAGGCCCCTAAGCTCCTG |
| | | ATCTACGGCGCATCCAACCTGGAAAGCGGAATCCCCTC |
| | | GCGGTTCTCGGGAAGCGGCTCTGGGACCGACTTCACCC |
| | | TTACTATCTCATCGGTGCAACCGGAGGACTTCGCCACCT |
| | | ACTACTGCCAACAGTCCCACGAATACCCATTCACCTTTG |
| | | GACAAGGCACTAAGCTGGAGATTAAG |
| SD- | 61 | EVQLVQSGAEVKKPGASVKVSCKASGYTFTSYDINWVRQ |
| 787077_VH | | APGQGLEWMGWIFPGDGTSKYAQKFQGRVTMTTDKSTST |
| | | AYMELSSLRSEDTAVYYCARRLGSHWGQGTTVTVSS |
| SD- | 62 | DIQLTQSPSSLSASVGDRVTITCKASQSVDSDGDSYMNWY |
| 787077_VL | | QQKPGKAPKLLIYGASNLESGVPSRFSGSGSGTDFTLTISSL |
| | | QPEDFATYYCQQSHEYPFTFGQGTKLEIK |
| SD- | 63 | GYTFTSYD |
| 787077_hCDR1 | | |
| SD- | 64 | IFPGDGTS |
| 787077_hCDR2 | | |
| SD- | 65 | ARRLGSH |
| 787077_hCDR3 | | |
| SD- | 66 | QSVDSDGDSY |
| 787077_1CDR1 | | |
| SD- | 67 | GAS |
| 787077_1CDR2 | | |
| SD- | 68 | QQSHEYPFT |
| 787077_1CDR3 | | |
| SD- | 69 | GAAGTGCAGCTCGTGCAATCCGGCGCCGAAGTCAAGAA |
| 787077_VH_ | | GCCTGGGGCCTCAGTGAAGGTGTCCTGCAAAGCATCGG |
| DNA | | GGTACACCTTCACGAGCTACGACATCAACTGGGTCCGC |
| | | CAAGCTCCGGGACAGGGTCTGGAGTGGATGGGCTGGAT |
| | | TTTTCCCGGCGACGGTACCAGCAAATACGCGCAGAAGT |
| | | TCCAGGGCAGAGTGACCATGACCACCGACAAGTCCACT |
| | | TCAACCGCCTACATGGAGCTGTCCTCCCTGCGGTCGGA |
| | | GGATACTGCCGTGTATTACTGTGCCCGGAGGCTTGGAA |
| | | GCCACTGGGGACAGGGAACTACTGTGACCGTGTCGTCC |
| SD- | 70 | GATATTCAGCTGACGCAGAGCCCCTCGTCCCTCTCCGCT |
| 787077_VL_ | | TCCGTGGGAGATCGCGTCACCATTACTTGCAAAGCCAG |
| DNA | | CCAGTCCGTGGACTCGGACGGAGACTCCTACATGAACT |
| | | GGTACCAGCAGAAGCCAGGAAAGGCCCCGAAGCTGCTT |
| | | ATCTACGGGGCCTCCAACTTGGAATCGGGAGTGCCTTC |
| | | ACGGTTCTCTGGTTCCGGCTCCGGCACTGACTTTACCCT |
| | | GACCATCAGCAGCCTGCAGCCGGAGGACTTCGCGACTT |
| | | ACTACTGCCAACAGTCACACGAATATCCCTTCACCTTCG |
| | | GCCAAGGGACCAAGCTGGAGATCAAG |
| SD- | 71 | EVQLVQSGAEVKKPGASVKVSCKASGYTFTSYDINWVRQ |
| 638526_VH | | APGQGLEWMGWIFPGDGTSKYAQKFQGRVTMTTDKSTST |
| | | AYMELSSLRSEDTAVYYCARRLGSHWGQGTTVTVSS |
| SD- | 72 | DIQLTQSPSSLSASVGDRATITCKASQSVDSDGDSYMNWY |
| 638526_VL | | QQKPGKAPKLLIYGASNLESGIPSRFSGSGSGTDFTLTISSV |
| | | QPEDFATYYCQQSHEYPFTFGQGTKLEIK |
| SD- | 73 | GYTFTSYD |
| 638526_hCDR1 | | |
| SD- | 74 | IFPGDGTS |
| 638526_hCDR2 | | |
| SD- | 75 | ARRLGSH |
| 638526_hCDR3 | | |
| SD- | 76 | QSVDSDGDSY |
| 638526_1CDR1 | | |
| SD- | 77 | GAS |
| 638526_1CDR2 | | |
| SD- | 78 | QQSHEYPFT |
| 638526_1CDR3 | | |
| SD- | 79 | GAAGTGCAGCTCGTGCAATCCGGCGCCGAAGTCAAGAA |
| 638526_VH_ | | GCCTGGGGCCTCAGTGAAGGTGTCCTGCAAAGCATCGG |
| DNA | | GGTACACCTTCACGAGCTACGACATCAACTGGGTCCGC |
| | | CAAGCTCCGGGACAGGGTCTGGAGTGGATGGGCTGGAT |
| | | TTTTCCCGGCGACGGTACCAGCAAATACGCGCAGAAGT |
| | | TCCAGGGCAGAGTGACCATGACCACCGACAAGTCCACT |
| | | TCAACCGCCTACATGGAGCTGTCCTCCCTGCGGTCGGA |
| | | GGATACTGCCGTGTATTACTGTGCCCGGAGGCTTGGAA |
| | | GCCACTGGGGACAGGGAACTACTGTGACCGTGTCGTCC |
| SD- | 80 | GATATTCAGTTGACCCAGTCCCCGAGCTCACTGTCCGCT |
| 638526_VL_ | | TCCGTGGGTGATCGCGCCACTATCACGTGTAAAGCGTC |
| DNA | | CCAGAGCGTCGACTCCGACGGGGACTCCTACATGAACT |
| | | GGTATCAGCAGAAGCCCGGAAAGGCCCCTAAGCTCCTG |
| | | ATCTACGGCGCATCCAACCTGGAAAGCGGAATCCCCTC |
| | | GCGGTTCTCGGGAAGCGGCTCTGGGACCGACTTCACCC |
| | | TTACTATCTCATCGGTGCAACCGGAGGACTTCGCCACCT |
| | | ACTACTGCCAACAGTCCCACGAATACCCATTCACCTTTG |
| | | GACAAGGCACTAAGCTGGAGATTAAG |
| SD- | 81 | EVQLVQSGAEVKKPGASVKLSCKASGYTFTSYDINWVRQ |
| 710726_VH | | APGQGLEWIGWIFPGDGTSKYAQKFQGRATLTTDKSTSTA |
| | | YMELSSLRSEDTAVYYCARRLGSHWGQGTTLTVSS |
| SD- | 82 | DIQLTQSPSSLSASVGDRVTITCKASQSVDSDGDSYMNWY |
| 710726_VL | | QQKPGKAPKLLIYGASNLESGVPSRFSGSGSGTDFTLTISSL |
| | | QPEDFATYYCQQSHEYPFTFGQGTKLEIK |
| SD- | 83 | GYTFTSYD |
| 710726_hCDR1 | | |
| SD- | 84 | IFPGDGTS |
| 710726_hCDR2 | | |
| SD- | 85 | ARRLGSH |
| 710726_hCDR3 | | |
| SD- | 86 | QSVDSDGDSY |
| 710726_1CDR1 | | |
| SD- | 87 | GAS |
| 710726_1CDR2 | | |
| SD- | 88 | QQSHEYPFT |
| 710726_1CDR3 | | |
| SD- | 89 | GAAGTGCAGTTGGTGCAGTCGGGAGCCGAAGTCAAGAA |
| 710726_VH_ | | GCCTGGAGCGTCCGTGAAGCTGAGCTGCAAGGCCTCAG |
| DNA | | GATACACTTTCACTTCATATGACATCAACTGGGTCAGAC |
| | | AGGCACCGGGCCAAGGACTGGAGTGGATTGGCTGGATC |
| | | TTTCCCGGGGATGGCACGAGCAAATACGCCCAGAAGTT |
| | | CCAGGGTAGAGCGACCCTGACCACCGACAAGTCCACTT |
| | | CGACCGCCTACATGGAACTCTCCTCGCTGCGCTCCGAG |
| | | GACACCGCCGTGTACTACTGTGCTCGGAGGCTTGGGTC |
| | | CCACTGGGGTCAAGGCACCACTCTCACCGTGTCCAGC |
| SD- | 90 | GATATTCAGCTGACGCAGAGCCCCTCGTCCCTCTCCGCT |
| 710726_VL_ | | TCCGTGGGAGATCGCGTCACCATTACTTGCAAAGCCAG |
| DNA | | CCAGTCCGTGGACTCGGACGGAGACTCCTACATGAACT |
| | | GGTACCAGCAGAAGCCAGGAAAGGCCCCGAAGCTGCTT |
| | | ATCTACGGGGCCTCCAACTTGGAATCGGGAGTGCCTTC |
| | | ACGGTTCTCTGGTTCCGGCTCCGGCACTGACTTTACCCT |
| | | GACCATCAGCAGCCTGCAGCCGGAGGACTTCGCGACTT |
| | | ACTACTGCCAACAGTCACACGAATATCCCTTCACCTTCG |
| | | GCCAAGGGACCAAGCTGGAGATCAAG |
| SD- | 91 | EVQLVQSGAEVKKPGASVKLSCKASGYTFTSYDINWVRQ |
| 577776_VH | | APGQGLEWIGWIFPGDGTSKYAQKFQGRATLTTDKSTSTA |
| | | YMELSSLRSEDTAVYYCARRLGSHWGQGTTLTVSS |
| SD- | 92 | DIQLTQSPSSLSASVGDRATITCKASQSVDSDGDSYMNWY |
| 577776_VL | | QQKPGKAPKLLIYGASNLESGIPSRFSGSGSGTDFTLTISSV |
| | | QPEDFATYYCQQSHEYPFTFGQGTKLEIK |
| SD- | 93 | GYTFTSYD |
| 577776_hCDR1 | | |
| SD- | 94 | IFPGDGTS |
| 577776_hCDR2 | | |
| SD- | 95 | ARRLGSH |
| 577776_hCDR3 | | |
| SD- | 96 | QSVDSDGDSY |
| 577776_1CDR1 | | |
| SD- | 97 | GAS |
| 577776_1CDR2 | | |
| SD- | 98 | QQSHEYPFT |
| 577776_1CDR3 | | |
| SD- | 99 | GAAGTGCAGTTGGTGCAGTCGGGAGCCGAAGTCAAGAA |
| 577776_VH_ | | GCCTGGAGCGTCCGTGAAGCTGAGCTGCAAGGCCTCAG |
| DNA | | GATACACTTTCACTTCATATGACATCAACTGGGTCAGAC |
| | | AGGCACCGGGCCAAGGACTGGAGTGGATTGGCTGGATC |
| | | TTTCCCGGGGATGGCACGAGCAAATACGCCCAGAAGTT |
| | | CCAGGGTAGAGCGACCCTGACCACCGACAAGTCCACTT |
| | | CGACCGCCTACATGGAACTCTCCTCGCTGCGCTCCGAG |
| | | GACACCGCCGTGTACTACTGTGCTCGGAGGCTTGGGTC |
| | | CCACTGGGGTCAAGGCACCACTCTCACCGTGTCCAGC |
| SD- | 100 | GATATTCAGTTGACCCAGTCCCCGAGCTCACTGTCCGCT |
| 577776_VL_ | | TCCGTGGGTGATCGCGCCACTATCACGTGTAAAGCGTC |
| DNA | | CCAGAGCGTCGACTCCGACGGGGACTCCTACATGAACT |
| | | GGTATCAGCAGAAGCCCGGAAAGGCCCCTAAGCTCCTG |
| | | ATCTACGGCGCATCCAACCTGGAAAGCGGAATCCCCTC |
| | | GCGGTTCTCGGGAAGCGGCTCTGGGACCGACTTCACCC |
| | | TTACTATCTCATCGGTGCAACCGGAGGACTTCGCCACCT |
| | | ACTACTGCCAACAGTCCCACGAATACCCATTCACCTTTG |
| | | GACAAGGCACTAAGCTGGAGATTAAG |
| SD- | 101 | QVQLVQSGAEVKKPGASVKVSCKASGYTFTSYDINWVRQ |
| 127612_VH | | APGQRLEWMGWIFPGDGTSKYNEKFKGRVTITRDTSAST |
| | | AYMELSSLRSEDTAVYYCARRLGSHWGQGTLVTVSS |
| SD- | 102 | DIVLTQSPDSLAVSPGERATISCKASQSVDSDGDSYMNWY |
| 127612_VL | | QQKPGQPPKLLIYGASNLESGVPDRFSGSGSGTDFTLTISR |
| | | VEAEDVAVYYCQQSHEYPFTFGGGTKLEIK |
| SD- | 103 | GYTFTSYD |
| 127612_hCDR1 | | |
| SD- | 104 | IFPGDGTS |
| 127612_hCDR2 | | |
| SD- | 105 | ARRLGSH |
| 127612_hCDR3 | | |
| SD- | 106 | QSVDSDGDSY |
| 127612_1CDR1 | | |
| SD- | 107 | GAS |
| 127612_1CDR2 | | |
| SD- | 108 | QQSHEYPFT |
| 127612_1CDR3 | | |
| SD- | 109 | CAAGTTCAGCTCGTGCAGAGTGGAGCAGAAGTGAAAAA |
| 127612_VH_ | | GCCAGGGGCTTCAGTTAAGGTAAGCTGTAAAGCCTCCG |
| DNA | | GGTATACATTCACATCATACGACATAAATTGGGTGAGG |
| | | CAAGCCCCGGGTCAGCGGCTGGAGTGGATGGGGTGGAT |
| | | TTTCCCCGGCGATGGGACTTCCAAGTACAATGAAAAGT |
| | | TCAAGGGGCGAGTGACAATCACTAGGGACACTTCCGCC |
| | | AGCACGGCTTACATGGAACTCAGCTCACTCAGAAGTGA |
| | | GGATACCGCGGTCTATTACTGTGCTCGCAGGCTGGGAT |
| | | CCCACTGGGGCCAAGGGACTCTGGTTACAGTCTCCTCC |
| SD- | 110 | GACATTGTACTCACCCAGAGTCCAGACAGCTTGGCCGT |
| 127612_VL_ | | CAGTCCAGGTGAGAGAGCCACCATTAGCTGCAAGGCAT |
| DNA | | CTCAGAGCGTGGATAGTGATGGCGATAGCTACATGAAC |
| | | TGGTACCAGCAGAAACCAGGCCAGCCACCTAAGCTCCT |
| | | CATCTATGGCGCCTCTAACCTTGAATCTGGAGTGCCCGA |
| | | CCGCTTTAGCGGTAGCGGCAGCGGCACAGATTTCACTTT |
| | | GACAATTAGTCGCGTGGAGGCCGAAGACGTGGCAGTCT |
| | | ACTACTGCCAGCAGAGCCACGAGTACCCATTCACATTC |
| | | GGGGGAGGGACAAAGTTAGAGATTAAG |
| SD- | 111 | QVQLVQSGAEVKKPGASVKVSCKASGYTFTSYDINWVRQ |
| 837152_VH | | APGQRLEWMGWIFPGDGTSKYNEKFKGRVTITRDTSAST |
| | | AYMELSSLRSEDTAVYYCARRLGSHWGQGTLVTVSS |
| SD- | 112 | DIVLTQSPASLAVSPGQRATITCKASQSVDSDGDSYMNWY |
| 837152_VL | | QQKPGQPPKLLIYGASNLESGVPARFSGSGSGTDFTLTINP |
| | | VEANDTANYYCQQSHEYPFTFGQGTKLEIK |
| SD- | 113 | GYTFTSYD |
| 837152_hCDR1 | | |
| SD- | 114 | IFPGDGTS |
| 837152_hCDR2 | | |
| SD- | 115 | ARRLGSH |
| 837152_hCDR3 | | |
| SD- | 116 | QSVDSDGDSY |
| 837152_1CDR1 | | |
| SD- | 117 | GAS |
| 837152_1CDR2 | | |
| SD- | 118 | QQSHEYPFT |
| 837152_1CDR3 | | |
| SD- | 119 | CAAGTTCAGCTCGTGCAGAGTGGAGCAGAAGTGAAAAA |
| 837152_VH_ | | GCCAGGGGCTTCAGTTAAGGTAAGCTGTAAAGCCTCCG |
| DNA | | GGTATACATTCACATCATACGACATAAATTGGGTGAGG |
| | | CAAGCCCCGGGTCAGCGGCTGGAGTGGATGGGGTGGAT |
| | | TTTCCCCGGCGATGGGACTTCCAAGTACAATGAAAAGT |
| | | TCAAGGGGCGAGTGACAATCACTAGGGACACTTCCGCC |
| | | AGCACGGCTTACATGGAACTCAGCTCACTCAGAAGTGA |
| | | GGATACCGCGGTCTATTACTGTGCTCGCAGGCTGGGAT |
| | | CCCACTGGGGCCAAGGGACTCTGGTTACAGTCTCCTCC |
| SD- | 120 | GATATCGTTCTGACCCAGAGTCCAGCGTCCTTGGCCGTG |
| 837152_VL_ | | TCTCCAGGCCAACGGGCTACAATAACATGCAAGGCCTC |
| DNA | | TCAGAGCGTGGACTCTGACGGCGATAGTTACATGAATT |
| | | GGTATCAGCAGAAGCCTGGACAGCCCCCCAAACTGTTG |
| | | ATTTACGGGGCAAGCAACCTGGAGAGTGGCGTGCCCGC |
| | | ACGTTTTTCAGGCTCAGGGAGCGGTACCGACTTTACACT |
| | | TACAATTAATCCCGTGGAGGCTAATGATACTGCCAACT |
| | | ACTACTGTCAGCAGAGCCACGAGTATCCTTTTACTTTTG |
| | | GCCAGGGAACCAAACTGGAAATCAAA |
| SD- | 121 | QVQLVQSGAEVKKPGASVKVSCKASGYTFTSYDINWVRQ |
| 649072_VH | | APGQRLEWMGWIFPGDGTSKYNEKFKGRVTITRDTSAST |
| | | AYMELSSLRSEDTAVYYCARRLGTVWGQGTLVTVSS |
| SD- | 122 | DIVLTQSPASLAVSPGQRATITCKASQSVDSDGDSYMNWY |
| 649072_VL | | QQKPGQPPKLLIYGASNLESGVPARFSGSGSGTDFTLTINP |
| | | VEANDTANYYCQQSHEYPFTFGQGTKLEIK |
| SD- | 123 | GYTFTSYD |
| 649072_hCDR1 | | |
| SD- | 124 | IFPGDGTS |
| 649072_hCDR2 | | |
| SD- | 125 | ARRLGTV |
| 649072_hCDR3 | | |
| SD- | 126 | QSVDSDGDSY |
| 649072_1CDR1 | | |
| SD- | 127 | GAS |
| 649072_1CDR2 | | |
| SD- | 128 | QQSHEYPFT |
| 649072_1CDR3 | | |
| SD- | 129 | CAAGTTCAGCTCGTGCAGAGTGGAGCAGAAGTGAAAAA |
| 649072_VH_ | | GCCAGGGGCTTCAGTTAAGGTAAGCTGTAAAGCCTCCG |
| DNA | | GGTATACATTCACATCATACGACATAAATTGGGTGAGG |
| | | CAAGCCCCGGGTCAGCGGCTGGAGTGGATGGGGTGGAT |
| | | TTTCCCCGGCGATGGGACTTCCAAGTACAATGAAAAGT |
| | | TCAAGGGGCGAGTGACAATCACTAGGGACACTTCCGCC |
| | | AGCACGGCTTACATGGAACTCAGCTCACTCAGAAGTGA |
| | | GGATACCGCGGTCTATTACTGTGCTCGCAGGCTGGGAA |
| | | CCGTTTGGGGCCAAGGGACTCTGGTTACAGTCTCCTCC |
| SD- | 130 | GATATCGTTCTGACCCAGAGTCCAGCGTCCTTGGCCGTG |
| 649072_VL_ | | TCTCCAGGCCAACGGGCTACAATAACATGCAAGGCCTC |
| DNA | | TCAGAGCGTGGACTCTGACGGCGATAGTTACATGAATT |
| | | GGTATCAGCAGAAGCCTGGACAGCCCCCCAAACTGTTG |
| | | ATTTACGGGGCAAGCAACCTGGAGAGTGGCGTGCCCGC |
| | | ACGTTTTTCAGGCTCAGGGAGCGGTACCGACTTTACACT |
| | | TACAATTAATCCCGTGGAGGCTAATGATACTGCCAACT |
| | | ACTACTGTCAGCAGAGCCACGAGTATCCTTTTACTTTTG |
| | | GCCAGGGAACCAAACTGGAAATCAAA |

In some embodiments, provided herein is an anti-EGFRvIII antibody, wherein the antibody comprises the amino acid sequences of the following three VH CDRs: SEQ ID NOs: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125, respectively.

In some embodiments, provided herein is an anti-EGFRvIII antibody, wherein the antibody comprises the amino acid sequences of the following three VL CDRs: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128, respectively.

In some embodiments, provided herein is an anti-EGFRvIII antibody, wherein the antibody comprises the amino acid sequences of VH comprising the amino acid sequence of any one of the following SEQ ID NOs: 1, 11, 21, 31, 41, 51, 61, 71, 81, 91, 101, 111, 121.

In some embodiments, provided herein is an anti-EGFRvIII antibody, wherein the antibody comprises the amino acid sequences of VL comprising the amino acid sequence of any one of the following SEQ ID NOs: 2, 12, 22, 32, 42, 52, 62, 72, 82, 92, 102, 112, 122.

In some embodiments, provided herein is an anti-EGFRvIII antibody, wherein the antibody comprises the amino acid sequences of the following pair of heavy and light chains: SEQ ID NO: 1 and 2, 11 and 12, 21 and 22, 31 and 32, 41 and 42, 51 and 52, 61 and 62, 71 and 72, 81 and 82, 91 and 92, 101 and 102, 111 and 112, 121 and 122, respectively.

In some embodiments, provided herein is an anti-EGFRvIII antibody, wherein the antibody heavy and light chains are encoded by a nucleic acid with at least 80%, 85%, 90%, 95, 96, 97, 98, 99% or 100% sequence identity to SEQ ID NOS: 9 and 10, 19 and 20, 29 and 30, 39 and 40, 49 and 50, 59 and 60, 69 and 70, 79 and 80, 89 and 90, 99 and 100, 109 and 110, 119 and 120, 129 and 130, respectively.

### Example 9. scFv-Fc Anti-EGFRvIII

In some embodiments, the disclosure provides for tandem scFv antibodies, with multiple anti-EGFRvIII binding sites. Tandem scFv-Fc antibodies of the disclosure are composed two or more scFv binding sites in tandem on each antibody arm, optionally linked by a linker, optionally a flexible linker. In some embodiments, a tandem scFV antibody has a total of four or more scFv binding sites in a single scFv-Fc formatted antibody.

The VH1 and the VL1 of each scFV1 may be connected by a linker, e.g., a flexible linker.

The VH2 and the VL2 of each scFV2 may be connected by a linker, e.g., a flexible linker.

The scFvs on each antibody arm may be connected by a linker, e.g., a flexible linker. An exemplary linker comprises the following amino acid sequence: GGGGSGGGGSGGGGS (SEQ ID NO: 131).

### Example 10. Therapeutic Anti-EGFRvIII Antibodies

In some embodiments, the anti-EGFRvIII antibodies provided herein are useful for the treatment of a cancer that expresses EGFRvIII, e.g., glioblastoma, head and neck squamous cell carcinoma, Non-Small Cell Lung Cancer-Squamous Cell Carcinoma (NSCLC-SCC), prostate cancer, breast cancer, and colon cancer.

### Example 11. Administration of Therapeutic Anti-EGFRvIII Antibodies

The in vivo administration of the therapeutic anti-EGFRvIII antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the anti-EGFRvIII antibodies of the disclosure are administered intravenously. In some embodiments, the anti-EGFRvIII antibodies of the disclosure are administered subcutaneously. Administration of the therapeutic anti-EGFRvIII antibodies may be performed with any suitable excipients, carriers, or other agents to provide suitable or improved tolerance, transfer, delivery, and the like.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-Epidermal Growth Factor Receptor version III (EGFRvIII) antibody or antigen binding domain thereof, wherein the antibody or antigen binding domain comprises:
a. a heavy chain variable domain (VH) complementarity determining region (CDR) 1, VH CDR2 and VH CDR3 comprising an amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; or 123, 124, 125, respectively; and
b. a light chain variable domain (VL) CDR1, VL CDR2 and VL CDR3 comprising an amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; or 126, 127, 128, respectively.

2. The antibody or binding domain of claim 1, wherein the antibody comprises a VH comprising the amino acid sequence of any one of the following SEQ ID NOS: 1, 11, 21, 31, 41, 51, 61, 71, 81, 91, 101, 111, or 121.

3. The antibody or binding domain of claim 1, wherein the antibody comprises a VL comprising the amino acid sequence of any one of the following SEQ ID NOS: 2, 12, 22, 32, 42, 52, 62, 72, 82, 92, 102, 112, or 122.

4. The antibody or binding domain of claim 1, wherein the antibody is a monoclonal antibody.

5. The antibody or binding domain of claim 1, wherein the antibody is a full-length antibody.

6. The antibody or binding domain of claim 1, wherein the antibody is an antibody fragment.

7. The antibody or binding domain of claim 5, wherein the antibody is fused to an Fc domain of any one of the following: human IgG1, human IgG2, human IgG3, and human IgG4.

8. The antibody or binding domain of claim 1, wherein the antibody heavy chain comprises an amino acid sequence with at least 80%, 85%, 90%, 95, 96, 97, 98, 99% or 100% sequence identity with SEQ ID NOS: 1, 11, 21, 31, 41, 51, 61, 71, 81, 91, 101, 111, or 121, or antibodies that comprises therewith.

9. The antibody or binding domain of claim 1, wherein the antibody light chain comprises as amino acid sequence with at least 80%, 85%, 90%, 95, 96, 97, 98, 99% or 100% sequence identity with SEQ ID NOS: 2, 12, 22, 32, 42, 52, 62, 72, 82, 92, 102, 112, or 122.

10. The antibody or binding domain of claim 1, wherein the antibody comprises a heavy chain variable domain and light chain variable domain: SEQ ID NO: 1 and 2, 11 and 12, 21 and 22, 31 and 32, 41 and 42, 51 and 52, 61 and 62, 71 and 72, 81 and 82, 91 and 92, 101 and 102, 111 and 112, or 121 and 122, respectively.

11. The antibody or binding domain of claim 1, wherein the antibody heavy chain is encoded by a nucleic acid and the antibody light chain is encoded by a nucleic acid with at least 80%, 85%, 90%, 95, 96, 97, 98, 99% or 100% sequence identity to SEQ ID NOS: 9 and 10, 19 and 20, 29 and 30, 39 and 40, 49 and 50, 59 and 60, 69 and 70, 79 and 80, 89 and 90, 99 and 100, 109 and 110, 119 and 120, or 129 and 130, respectively.

12. The antibody or binding domain of claim 1, wherein the antibody or binding domain is afucosylated.

13. The antibody or binding domain of claim 1, wherein the antibody or binding domain is produced in a bacteria, fungal, mammalian, insect, or plant cell.

14. The antibody or binding domain of claim 1, wherein the antibody or binding fragment thereof does not bind EGFR1.

15. A method of treating a disease in a subject in need thereof, comprising administering to the subject a therapeutically effective amount of the antibody or antigen-binding domain thereof, wherein the antibody or antigen-binding domain comprises:
a. a heavy chain variable domain (VH) complementarity determining region (CDR) 1, VH CDR2 and VH CDR3 comprising an amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; or 123, 124, 125, respectively; and
b. a light chain variable domain (VL) CDR1, VL CDR2 and VL CDR3 comprising an amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; or 126, 127, 128, respectively.

16. The method of claim 15, wherein the disease is a cancer.

17. The method of claim 15, wherein the disease is cancer selected from glioblastoma, head and neck squamous cell carcinoma, Non-Small Cell Lung Cancer-Squamous Cell Carcinoma (NSCLC-SCC), prostate cancer, breast cancer, and colon cancer, wherein the cancer expresses EGFRvIII.

18. The method of claim 16, wherein cancer cells of the cancer are killed by antibody-dependent cell cytotoxicity (ADCC).

19. The method of claim 15, wherein the subject is human.

20. A polynucleotide that comprises a nucleic acid sequence with at least 80%, 85%, 90%, 95, 96, 97, 98, 99%, or 100% sequence identity with SEQ ID NOS: 9 and 10, 19 and 20, 29 and 30, 39 and 40, 49 and 50, 59 and 60, 69 and 70, 79 and 80, 89 and 90, 99 and 100, 109 and 110, 119 and 120, or 129 and 130, respectively.

21. A vector comprising the polynucleotide of claim 20.

22. A host cell comprising the vector of claim 21.

23. A method of making an anti-EGFRvIII antibody comprising expressing in a cell a nucleic acid encoding an antibody of claims antibody of claim 1.
